(19) United States
(12) Patent Application Publication
(10) Pub. No.: US 2015/0366906 A1
(43) Pub. Date: Dec. 24, 2015

(54) ALKALINE DRINK

(71) Applicant: Richard Alexander SCHMOTTER, San Francisco, CA (US)

(72) Inventor: Richard Alexander SCHMOTTER, San Francisco, CA (US)

(21) Appl. No.: 14/649,464

(22) PCT Filed: Feb. 4, 2014
(86) PCT No.: PCT/US14/14715

§ 371 Date: Jun. 3, 2015

Related U.S. Application Data
(60) Provisional application No. 61/733,729, filed on Dec. 5, 2012.
(60) Provisional application No. 61/916,187, filed on Dec. 14, 2013.

(51) Int. Cl. A61K 33/42; A61K 36/886; A61K 36/88; A23L 2/68; A61K 33/00; A23L 2/56; A23L 2/02; A61K 33/14; A61K 47/22

(52) U.S. Cl. CPC A61K 33/42; A61K 33/14; A61K 36/886; A61K 36/88; A61K 47/22; A61K 33/00; A23L 2/56; A23L 2/02; A23L 2/68; A23V 2002/00

(57) ABSTRACT

Alkaline drink compositions buffered to have a neutral to alkaline pH and methods of treatment using the same.

### CROSS-REFERENCE

This application claims priority to U.S. Provisional Patent Application No. 61/733,729, filed Dec. 5, 2012 and U.S. Provisional Patent Application No. 61/916,187, filed Dec. 14, 2013.

### BACKGROUND

Dehydration is a decrease in total body water. While total body water is mostly present in the blood plasma, humans can lose water from any source in the body, either from blood volume or from tissue water. Blood volume can only be lost if sodium is lost, while tissue water is lost independent of sodium loss. Both mechanisms can be acting on a person simultaneously.

Symptoms of dehydration can include headache, hypotension, and dizziness or fainting. Thirst and discomfort are frequently the first signs, as well as skin dryness and appetite loss. Confusion, fatigue, negative mood are associated mental symptoms. Loss of physical performance can occur, as well as low endurance, elevated heart rate, and elevated body temperature. If untreated, dehydration can result in nausea, paresthesia, delirium, loss of consciousness, or even death.

Dehydration is a decrease in total body water. While total body water is mostly present in the blood plasma, humans can lose water from any source in the body, either from blood volume or from tissue water.

Symptoms of dehydration can include headache, hypotension, and dizziness or fainting. Thirst and discomfort are frequently the first signs, as well as skin dryness and appetite loss. Confusion, fatigue, negative mood are associated mental symptoms. Loss of physical performance can occur, as well as low endurance, elevated heart rate, and elevated body temperature. If untreated, dehydration can result in nausea, paresthesia, delirium, loss of consciousness, or even death.

There is a need in the art for new and improved drinks that prevent symptoms of dehydration, prevents dental caries disease, improves oxygen delivery in body, and buffers acidic metabolites. In some embodiments, compositions disclosed herein use a phosphate buffering system to achieve a composition having a neutral to alkaline pH (e.g., a pH of 7.4 (+/−0.3)).

Other sports drinks are acidic. The consumption of acidic beverages (below pH 5.5) demineralizes the teeth leading to dental caries. The acidic beverages also contribute to increased dietary acid load, causing increased physical fatigue, and negatively affecting overall health.

In some embodiments, compositions disclosed herein are stable at an alkaline pH of 7.4 (+/−0.3), which is beneficial for oral health and the use of phosphate buffer increases physical performance by increasing oxygen delivery to tissues and buffering acidic metabolites.

### SUMMARY OF THE INVENTION

Disclosed herein, in certain embodiments, are compositions comprising: (a) at least one alkaline buffering agent; (b) at least one acid source; (c) at least one carbohydrate source; (d) at least one nutrient source; and (e) at least one magnesium fortification; wherein the composition has pH greater than or equal to 7.0. In some embodiments, the composition has a pH between about 7.1 to about 7.7. In some embodiments, the composition has a pH of about 7.4. In some embodiments, the at least one alkaline buffering agent comprises monopotassium phosphate, dipotassium phosphate, tripotassium phosphate, monosodium phosphate, disodium phosphate, trisodium phosphate, potassium bicarbonate, sodium bicarbonate, or any combinations thereof. In some embodiments, the at least one alkaline buffering agent comprises dipotassium phosphate. In some embodiments, the at least one acid source comprises ascorbic acid, citric acid, acetic acid, carbonic acid, or any combination thereof. In some embodiments, the at least one acid source comprises ascorbic acid. In some embodiments, the at least one carbohydrate source comprises sucrose, glucose, maltose, dextrose, or any combinations thereof. In some embodiments, the at least one carbohydrate source comprises at least one low glycemic index carbohydrate source. In some embodiments, the at least one low glycemic index carbohydrate source comprises agave nectar, coconut nectar, coconut palm sugar, lucuma powder, honey, maple syrup, yacon syrup, date sugar, brown rice syrup, barley malt syrup, blackstrap molasses, cane juice, sorghum syrup, lactose, galactose, fructose, mogrosides (a.k.a. luo han guo or Siraitia grosvenorii extract), sugar alcohol, erythritol, mannitol, isomalt, lactitol, sorbitol, glycerol, xylitol, maltitol, HSH, arabitol, sweet dietary fiber, inulin, oligofructose, xylooligosaccharide, or any combinations thereof. In some embodiments, the at least one carbohydrate source comprises agave nectar. In some embodiments, the at least one nutrient source comprises Aloe vera inner leaf extract, coconut water, green tea, black tea, white tea, tea, fruit juice, vegetable juice, or any combination thereof. In some embodiments, the at least one nutrient source comprises Aloe vera inner leaf extract. In some embodiments, the at least one magnesium fortification comprises magnesium chloride, magnesium citrate, magnesium aspartate, magnesium lactate, magnesium glycinate, magnesium sulfate, magnesium oxide, magnesium hydroxide, or any combination thereof. In some embodiments, the at least one magnesium fortification comprises magnesium chloride. In some embodiments, the compositions further comprise at least one antioxidant selected from the group consisting of: ascorbic acid (Vitamin C), vitamin A, vitamin E, beta-carotene, other carotenoids, flavonoids, phenolic acids, curcumin, flavonoligans, xanthones, eugenol, capsaicin, bilirubin, citric acid, oxalic acid, phytic acid, uric acid, R-alpha-lipoic acid, N-Acetylcysteine, or any combination thereof. In some embodiments, the at least one antioxidant comprises ascorbic acid (Vitamin C). In some embodiments, the compositions further comprise a flavoring agent. In some embodiments, the at least one flavoring agent comprises grape, strawberry, acacia syrup, anise, apple, banana, berry, black currant, butterscotch, caramel, cherry, chocolate, cinnamon, eucalyptus, grapefruit, honey, lemon, licorice, lime, maple, marshmallow, menthol, mint, orange, peach, peppermint, raspberry, spearmint, strawberry, tangerine, vanilla, walnut, watermelon, wintergreen, acesulfame potassium, alitame, aspartame, cyclamate, neohesperidin DC, neotame, saccharin, sucralose, or any combinations thereof. In some embodiments, the at least one flavoring agent comprises at least one natural flavoring agent. In some embodiments, the composition is a beverage for human consumption. In some embodiments, the composition is a liquid. In some embodiments, the compositions comprise between about 2.5 mg/mL and about 3.5 mg/mL alkaline buffering agent. In some embodiments, the compositions comprise about 3 mg/mL alkaline buffering agent. In some embodiments, the compositions comprise between about 0.2 w/w % and about 0.4 w/w % alkaline buffering agent. In some embodiments, the compositions comprise about 0.3 w/w % alkaline buffering agent. In some embodiments, the compositions comprise between about 0.4 mg/mL and about 1.0 mg/mL acid source. In some embodiments, the compositions comprise about 0.6 mg/mL acid source. In some embodiments, the compositions comprise between about 0.04 w/w % and about 0.10 w/w % acid source. In some embodiments, the compositions comprise about 0.06 w/w % acid source. In some embodiments, the compositions comprise between about 50 mg/mL and about 70 mg/mL carbohydrate source. In some embodiments, the compositions comprise about 60 mg/mL carbohydrate source. In some embodiments, the compositions comprise between about 4 w/w % and about 10 w/w % carbohydrate source. In some embodiments, the compositions comprise about 6 w/w% carbohydrate source. In some embodiments, the compositions comprise between about 0.25 mg/mL and about 0.40 mg/mL nutrient source. In some embodiments, the compositions comprise about 0.3 mg/mL nutrient source. In some embodiments, the compositions comprise between about 0.01 w/w % and about 0.05 w/w % nutrient source. In some embodiments, the compositions comprise about 0.03 w/w % nutrient source. In some embodiments, the compositions comprise between about 0.1 mg/mL and about 0.5 mg/mL magnesium fortification. In some embodiments, the compositions comprise between about 0.01 w/w % and about 0.05 w/w % magnesium fortification. In some embodiments, the compositions comprise about 0.03 w/w % magnesium fortification. In some embodiments, the compositions comprise about 0.25 mg/mL magnesium fortification. In some embodiments, the compositions comprise between about 0.4 mg/mL and about 1.0 mg/mL antioxidant. In some embodiments, the compositions comprise about 0.6 mg/mL antioxidant. In some embodiments, the compositions comprise between about 0.04 w/w % and about 0.10 w/w % antioxidant. In some embodiments, the compositions comprise about 0.06 w/w % antioxidant. In some embodiments, the compositions comprise between about 2 mg/mL and about 5 mg/mL flavoring agent. In some embodiments, the compositions comprise about 3 mg/mL flavoring agent. In some embodiments, the compositions comprise between about 0.1 w/w % and about 0.6 w/w % flavoring agent. In some embodiments, the compositions comprise about 0.3 w/w % flavoring agent. In some embodiments, the composition is a solid. In some embodiments, the compositions comprise between about 4.0 w/w % and about 5.0 w/w % alkaline buffering agent. In some embodiments, the compositions comprise about 4.5 w/w % alkaline buffering agent. In some embodiments, the compositions comprise between about 75 w/w % and about 95 w/w % carbohydrate source. In some embodiments, the compositions comprise about 90 w/w % carbohydrate source. In some embodiments, the compositions comprise between about 0.3 w/w % and about 0.7 w/w % nutrient source. In some embodiments, the compositions comprise about 0.5 w/w % nutrient source. In some embodiments, the compositions comprise between about 0.2 w/w % and about 0.6 w/w % magnesium fortification. In some embodiments, the compositions comprise about 0.4 w/w % magnesium fortification. In some embodiments, the compositions comprise between about 0.5 w/w % and about 2.0 w/w % antioxidant. In some embodiments, the compositions comprise about 1 w/w % antioxidant. In some embodiments, the compositions comprise between about 3 w/w % and about 6 w/w % flavoring agent. In some embodiments, the compositions comprise about 4.5 w/w % flavoring agent. In some embodiments, the composition has a shelf life of greater than 300 days. In some embodiments, the composition has a shelf life of greater than 350 days. In some embodiments, the composition has a shelf life of greater than 400 days. In some embodiments, the composition has a shelf life of greater than 450 days.

Disclosed herein, in certain embodiments, are compositions comprising: (a) at least one alkaline buffering agent; (b) at least one acid source; and (c) at least one carbohydrate source; wherein the composition has pH greater than 7.0. In some embodiments, the compositions further comprise at least one of: a nutrient source, antioxidant, magnesium fortification, and a flavoring agent. In some embodiments, the composition has a pH between about 7.1 to about 7.7. In some embodiments, the composition has a pH of about 7.4. In some embodiments, the at least one alkaline buffering agent comprises monopotassium phosphate, dipotassium phosphate, tripotassium phosphate, monosodium phosphate, disodium phosphate, trisodium phosphate, potassium bicarbonate, sodium bicarbonate, or any combinations thereof. In some embodiments, the at least one alkaline buffering agent comprises dipotassium phosphate. In some embodiments, the at least one acid source comprises ascorbic acid, citric acid, acetic acid, carbonic acid, or any combination thereof. In some embodiments, the at least one acid source comprises ascorbic acid. In some embodiments, the at least one carbohydrate source comprises sucrose, glucose, maltose, dextrose, or any combinations thereof. In some embodiments, the at least one carbohydrate source comprises at least one low glycemic index carbohydrate source. In some embodiments, the at least one low glycemic index carbohydrate source comprises agave nectar, coconut nectar, coconut palm sugar, lucuma powder, honey, maple syrup, yacon syrup, date sugar, brown rice syrup, barley malt syrup, blackstrap molasses, cane juice, sorghum syrup, lactose, galactose, fructose, mogrosides (a.k.a. luo han guo or Siraitia grosvenorii extract), sugar alcohol, erythritol, mannitol, isomalt, lactitol, sorbitol, glycerol, xylitol, maltitol, HSH, arabitol, sweet dietary fiber, inulin, oligofructose, xylooligosaccharide, or any combinations thereof. In some embodiments, the at least one carbohydrate source comprises agave nectar. In some embodiments, the composition is a beverage for human consumption. In some embodiments, the composition is a liquid. In some embodiments, the compositions comprise between about 2.5 mg/mL and about 3.5 mg/mL alkaline buffering agent. In some embodiments, the compositions comprise about 3 mg/mL alkaline buffering agent. In some embodiments, the compositions comprise between about 0.2 w/w % and about 0.4 w/w % alkaline buffering agent. In some embodiments, the compositions comprise about 0.3 w/w % alkaline buffering agent. In some embodiments, the compositions comprise between about 0.4 mg/mL and about 1.0 mg/mL acid source. In some embodiments, the compositions comprise about 0.6 mg/mL acid source. In some embodiments, the compositions comprise between about 0.04 w/w % and about 0.10 w/w % acid source. In some embodiments, the compositions comprise about 0.06 w/w % acid source. In some embodiments, the compositions comprise between about 50 mg/mL and about 70 mg/mL carbohydrate source. In some embodiments, the compositions comprise about 60 mg/mL carbohydrate source. In some embodiments, the compositions comprise between about 4 w/w % and about 10 w/w % carbohydrate source. In some embodiments, the compositions comprise about 6 w/w % carbohydrate source. In some embodiments, the compositions comprise between about 0.25 mg/mL and about 0.40 mg/mL nutrient source. In some embodiments, the compositions comprise about 0.3 mg/mL nutrient source. In some embodiments, the compositions comprise between about 0.01 w/w % and about 0.05 w/w % nutrient source. In some embodiments, the compositions comprise about 0.03 w/w % nutrient source. In some embodiments, the compositions comprise between about 0.1 mg/mL and about 0.5 mg/mL magnesium fortification. In some embodiments, the compositions comprise between about 0.01 w/w % and about 0.05 w/w % magnesium fortification. In some embodiments, the compositions comprise about 0.03 w/w % magnesium fortification. In some embodiments, the compositions comprise about 0.25 mg/mL magnesium fortification. In some embodiments, the compositions comprise between about 0.4 mg/mL and about 1.0 mg/mL antioxidant. In some embodiments, the compositions comprise about 0.6 mg/mL antioxidant. In some embodiments, the compositions comprise between about 0.04 w/w % and about 0.10 w/w % antioxidant. In some embodiments, the compositions comprise about 0.06 w/w % antioxidant. In some embodiments, the compositions comprise between about 2 mg/mL and about 5 mg/mL flavoring agent. In some embodiments, the compositions comprise about 3 mg/mL flavoring agent. In some embodiments, the compositions comprise between about 0.1 w/w % and about 0.6 w/w % flavoring agent. In some embodiments, the compositions comprise about 0.3 w/w % flavoring agent. In some embodiments, the composition is a solid. In some embodiments, the compositions comprise between about 4.0 w/w % and about 5.0 w/w % alkaline buffering agent. In some embodiments, the compositions comprise about 4.5 w/w % alkaline buffering agent. In some embodiments, the compositions comprise between about 75 w/w % and about 95 w/w % carbohydrate source. In some embodiments, the compositions comprise about 90 w/w % carbohydrate source. In some embodiments, the compositions comprise between about 0.3 w/w % and about 0.7 w/w % nutrient source. In some embodiments, the compositions comprise about 0.5 w/w % nutrient source. In some embodiments, the compositions comprise between about 0.2 w/w % and about 0.6 w/w % magnesium fortification. In some embodiments, the compositions comprise about 0.4 w/w % magnesium fortification. In some embodiments, the compositions comprise between about 0.5 w/w % and about 2.0 w/w % antioxidant. In some embodiments, the compositions comprise about 1 w/w % antioxidant. In some embodiments, the compositions comprise between about 3 w/w % and about 6 w/w % flavoring agent. In some embodiments, the compositions comprise about 4.5 w/w % flavoring agent. In some embodiments, the composition has a shelf life of greater than 300 days. In some embodiments, the composition has a shelf life of greater than 350 days. In some embodiments, the composition has a shelf life of greater than 400 days. In some embodiments, the composition has a shelf life of greater than 450 days.

Disclosed herein, in certain embodiments, are methods of treating or preventing dehydration in an individual in need thereof, comprising administering to the individual a composition disclosed herein. In some embodiments, the dehydration is associated with prolonged physical activity, prolonged exposure to dry air, blood loss or hypotension (e.g., due to physical trauma), diarrhea, hyperthermia, shock, vomiting, burns, lacrimation, use of drugs (e.g., methamphetamines), excessive consumption of alcoholic beverages, infectious diseases (e.g, cholera, gastroenteritis, shigellosis, and yellow fever), malnutrition, electrolyte disturbance (hypernatremia, hyponatremia), fasting, hyperglycemia, glycosuria, uremia, diabetes insipidus, or any combination thereof.

Disclosed herein, in certain embodiments, are methods of alleviating one or more symptom(s) of dehydration in an individual in need thereof, comprising administering to the individual a composition disclosed herein. In some embodiments, the one or more symptom(s) comprises headache, hypotension, dizziness, dry or sticky mouth, sleepiness or tiredness, thirst, decreased sweating, decreased urination, constipation dry skin, rapid heartbeat, rapid breathing, fever, delirium, or any combination thereof.

### DETAILED DESCRIPTION OF THE INVENTION

The body is naturally alkaline, with the pH of blood being 7.365. This value is often referred to as physiological pH. An arterial pH below 7.35 is referred to as acidemia or acidosis. If arterial blood and extracellular fluid departs from safe ranges, cell damage may result.

Metabolic acidosis can result from the production of metabolic acids, or from decreases in acid excretion by the kidneys. Renal acidosis is usually associated with increased urea and creatinine Lactic acidosis can occur from insufficient oxygen diffusion into tissues from arterial blood. Increased acid production can also occur from starvation or diabetic acidosis.

Respiratory acidosis can result from a build-up of carbon dioxide in the blood due to hypoventilation. Pulmonary disease is the most common cause, and includes pneumothorax, emphysema, bronchitis, asthma, pneumonia, muscular dystrophy, and aspiration. Head injuries, brain tumors, anesthetics, and sedatives, can also lead to respiratory acidosis.

Negative health effects from acidosis include headache, sleepiness, confusion, loss of consciousness, coma, seizures, muscle weakness, diarrhea, shortness of breath, coughing, heart arrhythmia, increased heart rate, nausea, and vomiting.

Conventional drinks for rehydration are generally very acidic. Consuming acidic beverages leads to demineralization of the teeth and to dental caries and erosion. The mineral content of teeth is sensitive to acidity in the mouth. When the pH at the tooth surface reaches 5.5 or lower, demineralization of the tooth surface begins to occur. People with decreased saliva production are at increased risk due to the decreased pH buffering capacity of their saliva. These people can include patients with Sjogren's syndrome, diabetes mellitus, diabetes insipidus, or sarcoidosis, people using antihistamines, antidepressants, radiation therapy, tetrahydrocannabinol (THC), methamphetamine, tobacco, or any medication with a side effect of dry mouth.

Acidic beverages can also contribute to increased dietary acidic load and acidosis and lead to physical fatigue and decreased overall health, including kidney stones, kidney disease, weakening of bones or osteoporosis, weakening of joints or arthritis, weakening of muscles, heart disease, high blood pressure, and back pain.

By balancing the pH of a drink with an alkaline buffering system, the drink is already close to physiological pH, and the buffer can further neutralize acidic foods and metabolites. Additionally, such compositions can increase the amount of oxygen delivered to cells during exercise. Tooth decay and other dental problems related to acidity can be avoided, prevented, or diminished. It can help balance an acidic diet and counter negative health effects from acidosis. It can allow for more oxygen delivery to peripheral tissues via the blood. It can counter acid-induced muscular dystrophy.

As stated above, the compositions disclosed herein prevent dental caries disease, improves oxygen delivery in body, and buffers acidic metabolites. The compositions disclosed herein solve these problems associated with sports drinks.

The compositions disclosed herein are neutral to alkaline, utilizing an alkaline buffer (e.g., dipotassium phosphate buffer) to achieve a stable alkaline state (e.g., a pH of 7.4 (+/−0.3)). The pH prevents tooth demineralization and promotes healthy oral micro flora, decreasing dental caries disease. The use of dipotassium phosphate to stabilize the beverage at a neutral to alkaline pH causes alkalizing effects on the blood, acting on hemoglobin and allowing for increased oxygen delivery to tissues. The pH in combination with high potassium concentration causes increased bicarbonate secretion into the blood by parietal cells in the wall of the stomach. This causes neutralization and excretion of harmful acidic metabolites in the blood. The high phosphate concentration acts as a urinary buffer, allowing for increased acid excretion via the urinary system and long term control of acid imbalance (which is detrimental to physical performance and overall systemic health).

The compositions disclosed herein differ from those that currently exist. This is the first sports drink that is neutral to alkaline rather than acidic, preventing the oral and systemic problems that occur with acidic beverage consumption. This beverage also utilizes a phosphate buffering system to naturally increase the energy producing ability of cells and allow for more oxygen delivery to peripheral tissues.

This invention is an improvement on what currently exists. This is the first sports drink that is neutral to alkaline rather than acidic, preventing the oral and systemic problems that occur with acidic beverage consumption. This beverage also utilizes a phosphate buffering system shown to naturally increase 2, 3 Bisphosphoglycerate in the blood and allow for more oxygen delivery to peripheral tissues.

Acids contacting the teeth directly demineralize the teeth leading to cavity formation. Acidic beverages increase dietary acid load, leading to decreased physical performance and systemic health complications.

In some embodiments, the compositions disclosed herein are stable at an alkaline pH of 7.4 (+/−0.3), which is beneficial for oral health and the specific use of phosphate buffer increases physical performance.

### Alkaline Drink Compositions

Disclosed herein, in certain embodiments, are compositions including solutions, suspensions, syrups, powders, or tablets comprising (a) at least one alkaline pH buffering agent, (b) at least one acid source, and (c) at least one carbohydrate source. In some embodiments, the at least one alkaline pH buffering agent is dipotassium phosphate. In some embodiments, the at least one acid is ascorbic acid. In some embodiments, the at least one carbohydrate is agave nectar. In some embodiments, the alkaline pH buffering agent and the acid source comprise a buffering system.

The term “about” as used anywhere within this specification refers to the number value +/−10%, +/−9%, +/−8%, +/−7%, +/−6%, +/−5%, +/−4%, +/−3%, +/−2%, or +/−1%.

In some embodiments, the compositions disclosed herein comprise:
a. Dipotassium phosphate
b. Aloe vera inner leaf extract
c. agave nectar
d. magnesium chloride
e. ascorbic acid
f. natural flavors

The dipotassium phosphate acts as the buffering agent, which with the ascorbic acid forms a buffering system, allowing for an alkaline pH of 7.4 (+/−0.3). The Aloe vera inner leaf extract acts as a source of nutrients necessary for optimal health and performance. The agave nectar is the source of carbohydrate for optimal sustained energy with a low glycemic index. The magnesium chloride is for fortification of magnesium for increased nutrient absorption and proper cell function. The ascorbic acid is also for added antioxidant and increased immune function. The natural flavors are for flavor profile.

The alkaline buffering system (e.g., the phosphate and ascorbic acid) is necessary for achieving a stable alkaline pH (e.g., a pH of 7.4 (+/−0.3)). The Aloe vera inner leaf extract, agave nectar, magnesium chloride, and natural flavors are optional and variable.

Any ingredient may be manipulated in concentration to achieve a similar function. An additional ingredient may be added or a non-essential ingredient may be removed or altered. In some embodiments, the concentration of the phosphate buffer is modified in concentration, or grouped with another acid or base to elicit similar functionality at a pH greater than or less than 7.4.

The use of an alkaline (e.g., phosphate) buffer system to achieve a stable alkaline pH (e.g., 7.4 (+/−0.3)), in context of a rehydration beverage is unique and may have multiple benefits. The alkaline pH (e.g., 7.4 (+/−0.3)) raises the oral pH and may promote tooth remineralization. Further, in certain instances, the use of potassium as an alkaline buffer may enhance athletic performance. Additionally, The digestion of dipotassium phosphate in the stomach and small intestine. In the stomach, potassium is actively absorbed by the potassium/hydrogen exchange mechanism, producing bicarbonate ion and causing an “alkaline tide” and beneficial alkalization of the blood system (offsetting dietary and metabolic acidosis).

In certain instances, the use of potassium as an alkaline buffer increases oxygen delivery to muscles during exercise may improve power output and performance. Phosphate acts as a buffer and neutralizes acidic waste to maintain ion gradients and allow metabolism to continue. Additionally, metabolic processes occur by means of phosphorylation of metabolic intermediates (example AMP→cAMP, leading to phosphorylation cascade and propagation of a cellular signal) and end products (e.g., ADP gets phosphorylated into ATP by ATP synthase at the mitochondrial membrane, and energy is stored in that phosphate bond that will serve as the energy currency for other cellular processes to take place, for example muscle contraction). Additionally, increasing phosphate may increase the amount of 2,3 Bisphosphoglycerate (2,3 BPG) in circulation. 2,3 BPG is a naturally occurring phosphate compound that integrates into hemoglobin in red blood cells. The integration of 2,3 BPG has been shown to result in hemoglobin being maintained in a tense form rather than a relaxed form. The tense form of hemoglobin has a lower affinity for oxygen than the relaxed form, such that it delivers more oxygen to tissues under the equivalent circumstances than the relaxed form. This causes more oxygen to be delivered to muscles during exercise, and may result in an increase in athletic performance.

In some embodiments, the compositions disclosed herein do not have an aftertaste.

In some embodiments, the compositions disclosed herein have at least 3 components (e.g., an alkaline buffer, an acid source, and a carbohydrate source). In some embodiments, the compositions disclosed herein have at least 4 components (e.g., an alkaline buffer, an acid source, a carbohydrate source, and a nutrient source). In some embodiments, the compositions disclosed herein have at least 5 components (e.g., an alkaline buffer, an acid source, a carbohydrate source, a nutrient source, and a magnesium fortification). In some embodiments, the compositions disclosed herein have at least 6 components (e.g., an alkaline buffer, an acid source, a carbohydrate source, a nutrient source, a magnesium fortification, and an antioxidant). In some embodiments, the compositions disclosed herein have at least 7 components (e.g., an alkaline buffer, an acid source, a carbohydrate source, a nutrient source, a magnesium fortification, an antioxidant, and a flavoring). In some embodiments, the nutrient source and the carbohydrate source are not the same source. In some embodiments, the acid source and the antioxidant are the same.

In some embodiments, the compositions disclosed herein do not comprise artificial ingredients. In some embodiments, the compositions disclosed herein do not comprise preservatives. In some embodiments, the compositions disclosed herein do not comprise color additives. In some embodiments, the compositions disclosed herein do not comprise sodium.

### Alkaline pH Buffering Agents

Disclosed herein, in certain embodiments, are compositions including solutions, suspensions, syrups, powders, or tablets comprising (a) at least one alkaline pH buffering agent, (b) at least one acid source, and (c) at least one carbohydrate source.

Where the alkaline buffering agent is potassium-based, the potassium can serve as a dietary potassium supplement and aid in cardiovascular health. Decreased potassium (hypokalemia) can be caused by excessive potassium loss through urination, especially due to use of diuretic medicines. It can also be caused by chronic kidney disease, diabetic ketoacidosis, diarrhea, excessive use of laxatives, excessive sweating, vomiting, or primary aldosteronism. Symptoms of low potassium can include weakness, fatigue, muscle cramps, constipation, and heart arrhythmia.

Where the alkaline buffering system includes phosphate, the phosphate can act as a urinary buffer and allow for increased secretion of acid and long-term control of acid balance. Low levels of phosphate (hypophosphatemia) can be caused by alcohol abuse, refeeding syndrome, respiratory alkalosis, malabsorption of phosphate, blood cancers such as lymphoma and leukemia, liver failure, and tumor-induced osteomalacia. Symptoms of low phosphate can include rickets, muscle dysfunction and weakness, confusion, delirium, coma, dysfunction of white blood cells and decreased immune function, rhabdomyolysis, and hemolytic anemia.

In some embodiments, the at least one alkaline pH buffering agent is monopotassium phosphate, dipotassium phosphate, tripotassium phosphate, monosodium phosphate, disodium phosphate, trisodium phosphate, potassium bicarbonate, sodium bicarbonate, or any combination thereof. In some embodiments, the at least one alkaline pH buffering agent is dipotassium phosphate. In some embodiments, the at least one alkaline pH buffering agent is monopotassium phosphate. In some embodiments, the at least one alkaline pH buffering agent is tripotassium phosphate. In some embodiments, the at least one alkaline pH buffering agent is monosodium phosphate. In some embodiments, the at least one alkaline pH buffering agent is disodium phosphate. In some embodiments, the at least one alkaline pH buffering agent is trisodium phosphate. In some embodiments, the at least one alkaline pH buffering agent is potassium bicarbonate. In some embodiments, the at least one alkaline pH buffering agent is sodium bicarbonate.

In some embodiments, the composition is not acidic. In some embodiments, the composition has a pH of about 7.0 to about 9.0. In some embodiments, the composition has a pH of about 7.0 to about 8.5. In some embodiments, the composition has a pH of about 7.0 to about 8.0. In some embodiments, the composition has a pH of about 7.0 to about 7.5. In some embodiments, the composition has a pH of about 7.1 to about 7.7. In some embodiments, the composition has a pH of about 7.2 to about 7.6. In some embodiments, the composition has a pH of about 7.3 to about 7.5. In some embodiments, the composition has a pH of about 7.0. In some embodiments, the composition has a pH of about 7.1. In some embodiments, the composition has a pH of about 7.2. In some embodiments, the composition has a pH of about 7.3. In some embodiments, the composition has a pH of about 7.4. In some embodiments, the composition has a pH of about 7.5. In some embodiments, the composition has a pH of about 7.6. In some embodiments, the composition has a pH of about 7.7. In some embodiments, the composition or solution herein has a pH of at least 7.1, 7.2, 7.3, 7.4, 7.5, 7.6, 7.7, 7.8 7.9, or 8.0. In some embodiments, the composition has a pH of at least 7.0. In some embodiments, the composition has a pH of at least 7.1. In some embodiments, the composition has a pH of at least 7.2. In some embodiments, the composition has a pH of at least 7.3. In some embodiments, the composition has a pH of at least 7.4. In some embodiments, the composition has a pH of at least 7.5. In some embodiments, the composition has a pH of at least 7.6. In some embodiments, the composition has a pH of at least 7.7. In some embodiments, the composition has a pH of at least 7.8. In some embodiments, the composition has a pH of at least 7.9. In some embodiments, the composition has a pH of at least 8.0.

### Acid Sources

Disclosed herein, in certain embodiments, are compositions including solutions, suspensions, syrups, powders, or tablets comprising (a) at least one alkaline pH buffering agent, (b) at least one acid source, and (c) at least one carbohydrate source.

Any suitable acid source may be used. In some embodiments, the acid source comprises a single acid source. In some embodiments, the acid source comprises a combination of acid sources.

In some embodiments, the at least one acid source comprises ascorbic acid, citric acid, acetic acid, carbonic acid, or any combination thereof.

In some embodiments, the at least one acid source comprises ascorbic acid. In some embodiments, the at least one acid source comprises citric acid. In some embodiments, the at least one acid source comprises acetic acid. In some embodiments, the at least one acid source comprises carbonic acid.

### Carbohydrate Sources

Disclosed herein, in certain embodiments, are compositions including solutions, suspensions, syrups, powders, or tablets comprising (a) at least one alkaline pH buffering agent, (b) at least one acid source, and (c) at least one carbohydrate source. Any suitable carbohydrate source may be used. In some embodiments, the carbohydrate source comprises a single carbohydrate. In some embodiments, the carbohydrate source comprises a combination of carbohydrates. In some embodiments, the at least one carbohydrate source is a low glycemic index carbohydrate source.

In some embodiments, the at least one carbohydrate source comprises a carbohydrate selected from: sucrose, glucose, maltose, dextrose, and any combinations thereof. In some embodiments, the at least one carbohydrate source comprises sucrose. In some embodiments, the at least one carbohydrate source comprises glucose. In some embodiments, the at least one carbohydrate source comprises maltose. In some embodiments, the at least one carbohydrate source comprises dextrose. In some embodiments, the at least one carbohydrate source comprises fructose and glucose.

In some embodiments, the compositions disclosed herein have a sweetness rating of greater than 0.5. In some embodiments, the compositions disclosed herein have a sweetness rating of greater than 0.6. In some embodiments, the compositions disclosed herein have a sweetness rating of greater than 0.7. In some embodiments, the compositions disclosed herein have a sweetness rating of greater than 0.8. In some embodiments, the compositions disclosed herein have a sweetness rating of greater than 0.9. In some embodiments, the compositions disclosed herein have a sweetness rating of greater than 1. In some embodiments, the compositions disclosed herein have a sweetness rating between about 1 and about 2. In some embodiments, the compositions disclosed herein have a sweetness rating of greater than 1.1. In some embodiments, the compositions disclosed herein have a sweetness rating of greater than 1.2. In some embodiments, the compositions disclosed herein have a sweetness rating of greater than 1.3. In some embodiments, the compositions disclosed herein have a sweetness rating of greater than 1.4. In some embodiments, the compositions disclosed herein have a sweetness rating of greater than 1.5. In some embodiments, the compositions disclosed herein have a sweetness rating of greater than 1.6. In some embodiments, the compositions disclosed herein have a sweetness rating of greater than 1.7. In some embodiments, the compositions disclosed herein have a sweetness rating of greater than 1.8. In some embodiments, the compositions disclosed herein have a sweetness rating of greater than 1.9. In some embodiments, the compositions disclosed herein have a sweetness rating of greater than 2.

Low Glycemic Index Carbohydrate Sources

As used herein, “low glycemic index carbohydrate source” means a carbohydrate source having a glycemic index that is about 55 or less. Low glycemic index carbohydrates result in a slower and more stable effect on blood sugar levels when metabolized. This is beneficial for consumption by diabetic people. It can also prevent changes in energy level or mood caused by rapid changes in blood sugar.

In some embodiments, the at least one low glycemic index carbohydrate source is agave nectar. In some embodiments, the at least one low glycemic index carbohydrate source is coconut nectar. In some embodiments, the at least one low glycemic index carbohydrate source is coconut palm sugar. In some embodiments, the at least one low glycemic index carbohydrate source is lucuma powder. In some embodiments, the at least one low glycemic index carbohydrate source is honey. In some embodiments, the at least one low glycemic index carbohydrate source is maple syrup. In some embodiments, the at least one low glycemic index carbohydrate source is yacon syrup. In some embodiments, the at least one low glycemic index carbohydrate source is date sugar. In some embodiments, the at least one low glycemic index carbohydrate source is brown rice syrup. In some embodiments, the at least one low glycemic index carbohydrate source is barley malt syrup. In some embodiments, the at least one low glycemic index carbohydrate source is blackstrap molasses. In some embodiments, the at least one low glycemic index carbohydrate source is cane juice. In some embodiments, the at least one low glycemic index carbohydrate source is sorghum syrup. In some embodiments, the at least one low glycemic index carbohydrate source is mogroside (a.k.a. luo han guo or Siraitia grosvenorii extract).

In some embodiments, the at least one low glycemic index carbohydrate source is lactose. In some embodiments, the at least one low glycemic index carbohydrate source is galactose. In some embodiments, the at least one low glycemic index carbohydrate source is fructose.

In some embodiments, the at least one low glycemic index carbohydrate source is sugar alcohol. In some embodiments, the at least one low glycemic index carbohydrate source is erythritol. In some embodiments, the at least one low glycemic index carbohydrate source is mannitol. In some embodiments, the at least one low glycemic index carbohydrate source is isomalt. In some embodiments, the at least one low glycemic index carbohydrate source is lactitol. In some embodiments, the at least one low glycemic index carbohydrate source is sorbitol. In some embodiments, the at least one low glycemic index carbohydrate source is glycerol. In some embodiments, the at least one low glycemic index carbohydrate source is xylitol. In some embodiments, the at least one low glycemic index carbohydrate source is maltitol. In some embodiments, the at least one low glycemic index carbohydrate source is HSH. In some embodiments, the at least one low glycemic index carbohydrate source is arabitol.

In some embodiments, the at least one low glycemic index carbohydrate source is sweet dietary fiber. In some embodiments, the at least one low glycemic index carbohydrate source is inulin. In some embodiments, the at least one low glycemic index carbohydrate source is oligofructose.

In some embodiments, the at least one low glycemic index carbohydrate source is xylooligosaccharide.

In some embodiments, the at least one low glycemic index carbohydrate source is agave nectar, coconut nectar, coconut palm sugar, lucuma powder, honey, maple syrup, yacon syrup, date sugar, brown rice syrup, barley malt syrup, blackstrap molasses, cane juice, sorghum syrup, lactose, galactose, fructose, mogrosides (a.k.a. luo han guo or Siraitia grosvenorii extract), sugar alcohol, erythritol, mannitol, isomalt, lactitol, sorbitol, glycerol, xylitol, maltitol, HSH, arabitol, sweet dietary fiber, inulin, oligofructose, xylooligosaccharide, or any combinations thereof.

### Nutrient Source

In some embodiments, an alkaline drink composition disclosed herein further comprises at least one nutrient source. The nutrient source and the carbohydrate source need not be the same and are preferably not the same compound.

In some embodiments, the at least one nutrient source is Aloe vera inner leaf extract, coconut water, green tea, black tea, white tea, tea, fruit juice, vegetable juice, or any combination thereof.

In some embodiments, the at least one nutrient source is Aloe vera inner leaf extract. In some embodiments, the at least one nutrient source is coconut water. In some embodiments, the at least one nutrient source is green tea. In some embodiments, the at least one nutrient source is black tea. In some embodiments, the at least one nutrient source is white tea. In some embodiments, the at least one nutrient source is tea. In some embodiments, the at least one nutrient source is fruit juice. In some embodiments, the at least one nutrient source is vegetable juice.

In some embodiments, the at least one nutrient source comprises acemannan. The essential amino acids for humans are histidine, isoleucine, leucine, lysine, methionine, phenylalanine, threonine, tryptophan, and valine. In some embodiments, the at least one nutrient source comprises at least 1 of the essential amino acids. In some embodiments, the at least one nutrient source comprises at least 2 of the essential amino acids. In some embodiments, the at least one nutrient source comprises at least 3 of the essential amino acids. In some embodiments, the at least one nutrient source comprises at least 4 of the essential amino acids. In some embodiments, the at least one nutrient source comprises at least 5 of the essential amino acids. In some embodiments, the at least one nutrient source comprises at least 6 of the essential amino acids. In some embodiments, the at least one nutrient source comprises at least 7 of the essential amino acids. In some embodiments, the at least one nutrient source comprises at least 8 of the essential amino acids. In some embodiments, the at least one nutrient source comprises all 9 of the essential amino acids.

### Magnesium Fortification

In some embodiments, an alkaline drink composition disclosed herein further comprises at least one magnesium fortification. Magnesium aids in absorption of minerals such as calcium, contraction and relaxation of muscles, function of certain enzymes in the body, production and transport of energy, and protein production. Lack of magnesium, or magnesium deficiency, can result in anorexia, apathy, confusion, fatigue, hyper-excitability, insomnia, irritability, muscle twitches, muscle weakness, reduced memory function, reduced learning ability, or sleepiness. Magnesium deficiency can occur in people due to alcohol abuse, burns, certain medications, difficulty absorbing nutrients from the intestinal tract, low blood calcium levels, or surgery.

In some embodiments, the magnesium fortification is magnesium chloride, magnesium citrate, magnesium aspartate, magnesium lactate, magnesium glycinate, magnesium sulfate, magnesium oxide, magnesium hydroxide, or any combination thereof.

In some embodiments, the magnesium fortification is magnesium chloride. In some embodiments, the magnesium fortification is magnesium citrate. In some embodiments, the magnesium fortification is magnesium aspartate. In some embodiments, the magnesium fortification is magnesium lactate. In some embodiments, the magnesium fortification is magnesium glycinate. In some embodiments, the magnesium fortification is magnesium sulfate. In some embodiments, the magnesium fortification is magnesium oxide. In some embodiments, the magnesium fortification is magnesium hydroxide.

### Antioxidants

In some embodiments, an alkaline drink composition disclosed herein further comprises at least one antioxidant. Antioxidants are molecules that prevent cell damage against free radicals. They are critical in maintaining health. Consumption of adequate amounts of antioxidants can reduce risks of diseases including cancers and cardiovascular diseases. Without sufficient antioxidants, free radicals can lead to damage to DNA, lipids, and proteins.

In some embodiments, the at least one antioxidant is ascorbic acid (Vitamin C), vitamin A, vitamin E, beta-carotene, other carotenoids, flavonoids, phenolic acids, curcumin, flavonoligans, xanthones, eugenol, capsaicin, bilirubin, citric acid, oxalic acid, phytic acid, uric acid, R-alpha-lipoic acid, N-Acetylcysteine, or any combination thereof.

In some embodiments, the at least one antioxidant is ascorbic acid. In some embodiments, the at least one antioxidant is vitamin A. In some embodiments, the at least one antioxidant is vitamin E. In some embodiments, the at least one antioxidant is beta-carotene. In some embodiments, the at least one antioxidant are other carotenoids. In some embodiments, the at least one antioxidant are flavonoids. In some embodiments, the at least one antioxidant are phenolic acids. In some embodiments, the at least one antioxidant is curcumin. In some embodiments, the at least one antioxidant are flavonoligans. In some embodiments, the at least one antioxidant are xanthones. In some embodiments, the at least one antioxidant is eugenol. In some embodiments, the at least one antioxidant is capsaicin. In some embodiments, the at least one antioxidant is bilirubin. In some embodiments, the at least one antioxidant is citric acid. In some embodiments, the at least one antioxidant is oxalic acid. In some embodiments, the at least one antioxidant is phytic acid. In some embodiments, the at least one antioxidant is uric acid. In some embodiments, the at least one antioxidant is R-alpha-lipoic acid. In some embodiments, the at least one antioxidant is N-Acetylcysteine.

### Flavors

In some embodiments, an alkaline drink composition disclosed herein further comprises at least one flavoring agent. This at least one flavoring agent may include any flavoring agents approved by the FDA or Generally Recognized as Safe (GRAS) or any combination thereof.

In some embodiments, the flavoring agent is a natural flavoring agent.

In some embodiments, the flavoring agent is grape, strawberry, acacia syrup, anise, apple, banana, berry, black currant, butterscotch, caramel, cherry, chocolate, cinnamon, eucalyptus, grapefruit, honey, lemon, licorice, lime, maple, marshmallow, menthol, mint, orange, peach, peppermint, raspberry, spearmint, strawberry, tangerine, vanilla, walnut, watermelon, wintergreen, acesulfame potassium, alitame, aspartame, cyclamate, neohesperidin DC, neotame, saccharin, sucralose, or any combinations thereof.

In some embodiments, the flavoring agent is acesulfame potassium. In some embodiments, the flavoring agent is alitame. In some embodiments, the flavoring agent is aspartame. In some embodiments, the flavoring agent is cyclamate. In some embodiments, the flavoring agent is neohesperidin DC. In some embodiments, the flavoring agent is neotame. In some embodiments, the flavoring agent is saccharin. In some embodiments, the flavoring agent is sucralose. In some embodiments, the at least one flavoring agent is grape. In some embodiments, the at least one flavoring agent is strawberry. In some embodiments, the at least one flavoring agent is acacia syrup. In some embodiments, the at least one flavoring agent is anise. In some embodiments, the at least one flavoring agent is apple. In some embodiments, the at least one flavoring agent is banana. In some embodiments, the at least one flavoring agent is berry. In some embodiments, the at least one flavoring agent is black currant. In some embodiments, the at least one flavoring agent is butterscotch. In some embodiments, the at least one flavoring agent is caramel. In some embodiments, the at least one flavoring agent is cherry. In some embodiments, the at least one flavoring agent is chocolate. In some embodiments, the at least one flavoring agent is cinnamon. In some embodiments, the at least one flavoring agent is eucalyptus. In some embodiments, the at least one flavoring agent is grapefruit. In some embodiments, the at least one flavoring agent is honey. In some embodiments, the at least one flavoring agent is lemon. In some embodiments, the at least one flavoring agent is licorice. In some embodiments, the at least one flavoring agent is lime. In some embodiments, the at least one flavoring agent is maple. In some embodiments, the at least one flavoring agent is marshmallow. In some embodiments, the at least one flavoring agent is menthol. In some embodiments, the at least one flavoring agent is mint. In some embodiments, the at least one flavoring agent is orange. In some embodiments, the at least one flavoring agent is pear. In some embodiments, the at least one flavoring agent is peach. In some embodiments, the at least one flavoring agent is peppermint. In some embodiments, the at least one flavoring agent is raspberry. In some embodiments, the at least one flavoring agent is spearmint. In some embodiments, the at least one flavoring agent is strawberry. In some embodiments, the at least one flavoring agent is tangerine. In some embodiments, the at least one flavoring agent is vanilla. In some embodiments, the at least one flavoring agent is walnut. In some embodiments, the at least one flavoring agent is watermelon. In some embodiments, the at least one flavoring agent is wintergreen.

### Physical Form of Composition

In some embodiments, the composition is formulated as a solid dosage form (e.g., a tablet or a powder) or a non-solid dosage form (e.g., a solution, syrup, or suspension). In some embodiments, the composition is a solution. In some embodiments, the composition is a syrup. In some embodiments, the composition is a suspension. In some embodiments, the composition is a tablet. In some embodiments, the composition is a suspension tablet, a fast-melt tablet, a bite-disintegration tablet, a rapid-disintegration tablet, an effervescent tablet, or a caplet. In other embodiments, the composition is a powder. In some embodiments, the composition is a sterile packaged powder, a dispensable powder, or an effervescent powder.

In some embodiments, the composition has a shelf life greater than 300 days. In some embodiments, the composition has a shelf life greater than 350 days. In some embodiments, the composition has a shelf life of greater than 400 days. In some embodiments, the composition has a shelf life of greater than 450 days. In some embodiments, the composition has a shelf life of 450 days. In some embodiments, the composition has a shelf life between about 300 and about 500 days. In some embodiments, the composition has a shelf life between about 350 days and about 450 days. In some embodiments, the composition has a shelf life between about 400 days and about 500 days.

Non-Solid Dosage Forms

In some embodiments, the composition is a non-solid dosage form (e.g., a solution) with ready-to-consume concentrations of ingredients. In some embodiments, the composition is a concentrate (e.g., a syrup) which, when mixed together in appropriate amounts with water, achieves a final concentration of all ingredients within consumable and palatable levels.

In some embodiments, the non-solid dosage form is transparent. In some embodiments, the non-solid dosage form is translucent.

In some embodiments, the non-solid dosage form has a viscosity greater than or equal to about 0.9 cP at 25° C. In some embodiments, the non-solid dosage form has a viscosity greater than or equal to about 1 cP at 25° C. In some embodiments, the non-solid dosage form has a viscosity greater than or equal to about 1.5 cP at 25° C. In some embodiments, the non-solid dosage form has a viscosity greater than or equal to about 2 cP at 25° C. In some embodiments, the non-solid dosage form has a viscosity greater than or equal to about 2.5 cP at 25° C. In some embodiments, the non-solid dosage form has a viscosity greater than or equal to about 3 cP at 25° C. In some embodiments, the non-solid dosage form has a viscosity greater than or equal to about 3.5 cP at 25° C. In some embodiments, the non-solid dosage form has a viscosity greater than or equal to about 4 cP at 25° C. In some embodiments, the non-solid dosage form has a viscosity greater than or equal to about 4.5 cP at 25° C. In some embodiments, the non-solid dosage form has a viscosity greater than or equal to about 5 cP at 25° C.

In some embodiments, the non-solid dosage form has a dissolved solids content of less than 30 degrees Brix. In some embodiments, the non-solid dosage form has a dissolved solids content of less than 25 degrees Brix. In some embodiments, the non-solid dosage form has a dissolved solids content of less than 20 degrees Brix. In some embodiments, the non-solid dosage form has a dissolved solids content of less than 15 degrees Brix. In some embodiments, the non-solid dosage form has a dissolved solids content of less than 10 degrees Brix. In some embodiments, the non-solid dosage form has a dissolved solids content of less than 8 degrees Brix. In some embodiments, the non-solid dosage form has a dissolved solids content of less than 6 degrees Brix. In some embodiments, the non-solid dosage form has a dissolved solids content of less than 5 degrees Brix. In some embodiments, the non-solid dosage form has a dissolved solids content of less than 4 degrees Brix. In some embodiments, the non-solid dosage form has a dissolved solids content of less than 3 degrees Brix. In some embodiments, the non-solid dosage form has a dissolved solids content of less than 2 degrees Brix. In some embodiments, the non-solid dosage form has a dissolved solids content of less than 1 degree Brix.

In some embodiments, the composition comprises between about 1.5 mg/mL and about 6.0 mg/mL alkaline buffering agent (e.g., dipotassium phosphate). In some embodiments, the composition comprises between about 2.0 mg/mL and about 4.5 mg/mL alkaline buffering agent (e.g., dipotassium phosphate). In some embodiments, the composition comprises between about 2.5 mg/mL and about 3.5 mg/mL alkaline buffering agent (e.g., dipotassium phosphate). In some embodiments, the composition comprises between about 3.0 mg/mL and about 3.2 mg/mL alkaline buffering agent (e.g., dipotassium phosphate). In some embodiments, the composition comprises about 3.1 mg/mL alkaline buffering agent (e.g., dipotassium phosphate).

In some embodiments, the composition comprises about 2.5 mg/mL alkaline buffering agent (e.g., dipotassium phosphate). In some embodiments, the composition comprises about 2.6 mg/mL alkaline buffering agent (e.g., dipotassium phosphate). In some embodiments, the composition comprises about 2.7 mg/mL alkaline buffering agent (e.g., dipotassium phosphate). In some embodiments, the composition comprises about 2.8 mg/mL alkaline buffering agent (e.g., dipotassium phosphate). In some embodiments, the composition comprises about 2.9 mg/mL alkaline buffering agent (e.g., dipotassium phosphate). In some embodiments, the composition comprises about 3.0 mg/mL alkaline buffering agent (e.g., dipotassium phosphate). In some embodiments, the composition comprises about 3.1 mg/mL alkaline buffering agent (e.g., dipotassium phosphate). In some embodiments, the composition comprises about 3.2 mg/mL alkaline buffering agent (e.g., dipotassium phosphate). In some embodiments, the composition comprises about 3.3 mg/mL alkaline buffering agent (e.g., dipotassium phosphate). In some embodiments, the composition comprises about 3.4 mg/mL alkaline buffering agent (e.g., dipotassium phosphate). In some embodiments, the composition comprises about 3.5 mg/mL alkaline buffering agent (e.g., dipotassium phosphate).

In some embodiments, the composition comprises between about 0.15 w/w % and about 0.60 w/w % alkaline buffering agent (e.g., dipotassium phosphate). In some embodiments, the composition comprises between about 0.15 w/w % and about 0.60 w/w % alkaline buffering agent (e.g., dipotassium phosphate). In some embodiments, the composition comprises between about 0.20 w/w % and about 0.50 w/w % alkaline buffering agent (e.g., dipotassium phosphate). In some embodiments, the composition comprises between about 0.20 w/w % and about 0.40 w/w % alkaline buffering agent (e.g., dipotassium phosphate). In some embodiments, the composition comprises between about 0.25 w/w % and about 0.35 w/w % alkaline buffering agent (e.g., dipotassium phosphate).

In some embodiments, the composition comprises about 0.25 w/w % alkaline buffering agent (e.g. dipotassium phosphate). In some embodiments, the composition comprises about 0.26 w/w % alkaline buffering agent (e.g. dipotassium phosphate). In some embodiments, the composition comprises about 0.27 w/w % alkaline buffering agent (e.g. dipotassium phosphate). In some embodiments, the composition comprises about 0.28 w/w % alkaline buffering agent (e.g. dipotassium phosphate). In some embodiments, the composition comprises about 0.29 w/w % alkaline buffering agent (e.g. dipotassium phosphate). In some embodiments, the composition comprises about 0.30 w/w % alkaline buffering agent (e.g. dipotassium phosphate). In some embodiments, the composition comprises about 0.31 w/w % alkaline buffering agent (e.g. dipotassium phosphate). In some embodiments, the composition comprises about 0.32 w/w % alkaline buffering agent (e.g. dipotassium phosphate). In some embodiments, the composition comprises about 0.33 w/w % alkaline buffering agent (e.g. dipotassium phosphate). In some embodiments, the composition comprises about 0.34 w/w % alkaline buffering agent (e.g. dipotassium phosphate). In some embodiments, the composition comprises about 0.35 w/w % alkaline buffering agent (e.g. dipotassium phosphate).

In some embodiments, the composition comprises about 30 mg/mL carbohydrate source (e.g., agave nectar) to about 120 mg/mL carbohydrate source (e.g., agave nectar). In some embodiments, the composition comprises between about 40 mg/mL carbohydrate source (e.g., agave nectar) and about 100 mg/mL carbohydrate source (e.g., agave nectar). In some embodiments, the composition comprises between about 50 mg/mL carbohydrate source (e.g., agave nectar) and about 80 mg/mL carbohydrate source (e.g., agave nectar). In some embodiments, the composition comprises between about 55 mg/mL carbohydrate source (e.g., agave nectar) and about 65 mg/mL carbohydrate source (e.g., agave nectar). In some embodiments, the composition comprises between about 58 mg/mL carbohydrate source (e.g., agave nectar) and about 63 mg/mL carbohydrate source (e.g., agave nectar). In some embodiments, the composition comprises between about 60 mg/mL carbohydrate source (e.g., agave nectar) and about 62 mg/mL carbohydrate source (e.g., agave nectar). In some embodiments, the composition comprises about 61 mg/mL carbohydrate source (e.g., agave nectar).

In some embodiments, the composition comprises about 55 mg/mL carbohydrate source (e.g., agave nectar). In some embodiments, the composition comprises about 56 mg/mL carbohydrate source (e.g., agave nectar). In some embodiments, the composition comprises about 57 mg/mL carbohydrate source (e.g., agave nectar). In some embodiments, the composition comprises about 58 mg/mL carbohydrate source (e.g., agave nectar). In some embodiments, the composition comprises about 59 mg/mL carbohydrate source (e.g., agave nectar). In some embodiments, the composition comprises about 60 mg/mL carbohydrate source (e.g., agave nectar). In some embodiments, the composition comprises about 61 mg/mL carbohydrate source (e.g., agave nectar). In some embodiments, the composition comprises about 62 mg/mL carbohydrate source (e.g., agave nectar). In some embodiments, the composition comprises about 63 mg/mL carbohydrate source (e.g., agave nectar). In some embodiments, the composition comprises about 64 mg/mL carbohydrate source (e.g., agave nectar). In some embodiments, the composition comprises about 65 mg/mL carbohydrate source (e.g., agave nectar).

In some embodiments, the composition comprises between about 3 w/w % and about 12 w/w % carbohydrate source (e.g., agave nectar). In some embodiments, the composition comprises between about 4 w/w % and about 10 w/w % carbohydrate source (e.g., agave nectar). In some embodiments, the composition comprises between about 5 w/w % and about 8 w/w % carbohydrate source (e.g., agave nectar).

In some embodiments, the composition comprises about 5 w/w % carbohydrate source (e.g., agave nectar). In some embodiments, the composition comprises about 5.5 w/w % carbohydrate source (e.g., agave nectar). In some embodiments, the composition comprises about 6 w/w % carbohydrate source (e.g., agave nectar). In some embodiments, the composition comprises about 6.5 w/w % carbohydrate source (e.g., agave nectar). In some embodiments, the composition comprises about 7 w/w % carbohydrate source (e.g., agave nectar). In some embodiments, the composition comprises about 7.5 w/w % carbohydrate source (e.g., agave nectar). In some embodiments, the composition comprises about 8 w/w % carbohydrate source (e.g., agave nectar).

In some embodiments, the composition comprises carbohydrate source which comprises about 85% fructose and 15% dextrose. In some embodiments, the composition comprises carbohydrate source which comprises about 80% fructose and 20% dextrose. In some embodiments, the composition comprises carbohydrate source which comprises about 90% fructose and 10% dextrose.

In some embodiments, the composition comprises less than 25 grams of total sugar per 8 oz serving. In some embodiments, the composition comprises less than 20 grams of total sugar per 8 oz serving. In some embodiments, the composition comprises less than 15 grams of total sugar per 8 oz serving. In some embodiments, the composition comprises less than 10 grams of total sugar per 8 oz serving. In some embodiments, the composition comprises less than 9 grams of total sugar per 8 oz serving. In some embodiments, the composition comprises less than 8 grams of total sugar per 8 oz serving. In some embodiments, the composition comprises less than 7 grams of total sugar per 8 oz serving. In some embodiments, the composition comprises less than 6 grams of total sugar per 8 oz serving. In some embodiments, the composition comprises less than 5 grams of total sugar per 8 oz serving. In some embodiments, the composition comprises less than 4 grams of total sugar per 8 oz serving. In some embodiments, the composition comprises less than 3 grams of total sugar per 8 oz serving. In some embodiments, the composition comprises less than 2 grams of total sugar per 8 oz serving. In some embodiments, the composition comprises less than 1 gram of total sugar per 8 oz serving.

In some embodiments, the composition comprises between about 0.3 mg/mL and about 1.20 mg/mL acid source (e.g., ascorbic acid). In some embodiments, the composition comprises between about 0.4 mg/mL and about 1.0 mg/mL acid source (e.g., ascorbic acid). In some embodiments, the composition comprises between about 0.5 mg/mL and about .80 mg/mL acid source (e.g., ascorbic acid). In some embodiments, the composition comprises between about 0.6 and about 0.7 mg/mL acid source (e.g., ascorbic acid). In some embodiments, the composition comprises between about 0.6 and about 0.65 mg/mL acid source (e.g., ascorbic acid). In some embodiments, the composition comprises about 0.61 mg/mL acid source (e.g., ascorbic acid).

In some embodiments, the composition comprises about 0.55 mg/mL acid source (e.g., ascorbic acid). In some embodiments, the composition comprises about 0.56 mg/mL acid source (e.g., ascorbic acid). In some embodiments, the composition comprises about 0.57 mg/mL acid source (e.g., ascorbic acid). In some embodiments, the composition comprises about 0.58 mg/mL acid source (e.g., ascorbic acid). In some embodiments, the composition comprises about 0.59 mg/mL acid source (e.g., ascorbic acid). In some embodiments, the composition comprises about 0.60 mg/mL acid source (e.g., ascorbic acid). In some embodiments, the composition comprises about 0.61 mg/mL acid source (e.g., ascorbic acid). In some embodiments, the composition comprises about 0.62 mg/mL acid source (e.g., ascorbic acid). In some embodiments, the composition comprises about 0.63 mg/mL acid source (e.g., ascorbic acid). In some embodiments, the composition comprises about 0.64 mg/mL acid source (e.g., ascorbic acid). In some embodiments, the composition comprises about 0.65 mg/mL acid source (e.g., ascorbic acid).

In some embodiments, the composition comprises between about 0.03 w/w % and about 0.12 w/w % acid source (e.g., ascorbic acid). In some embodiments, the composition comprises between about 0.03 w/w % and about 0.12 w/w % acid source (e.g., ascorbic acid). In some embodiments, the composition comprises between about 0.04 w/w % and about 0.10 w/w % acid source (e.g., ascorbic acid). In some embodiments, the composition comprises between about 0.05 w/w % and about 0.08 w/w % acid source (e.g., ascorbic acid).

In some embodiments, the composition comprises about 0.05 w/w % acid source (e.g., ascorbic acid). In some embodiments, the composition comprises about 0.06 w/w % acid source (e.g., ascorbic acid). In some embodiments, the composition comprises about 0.07 w/w % acid source (e.g., ascorbic acid). In some embodiments, the composition comprises about 0.08 w/w % acid source (e.g., ascorbic acid).

In some embodiments, the composition comprises between about 0.15 mg/mL and about 0.60 mg/mL nutrient source (e.g., Aloe vera inner leaf extract). In some embodiments, the composition comprises between about 0.20 mg/mL and about 0.50 mg/mL nutrient source (e.g., Aloe vera inner leaf extract). In some embodiments, the composition comprises between about 0.25 mg/mL and about 0.40 mg/mL nutrient source (e.g., Aloe vera inner leaf extract). In some embodiments, the composition comprises between about 0.25 mg/mL and about 0.35 mg/mL nutrient source (e.g., Aloe vera inner leaf extract). In some embodiments, the composition comprises about 0.30 mg/mL nutrient source (e.g., Aloe vera inner leaf extract).

In some embodiments, the composition comprises about 0.25 mg/mL nutrient source (e.g., Aloe vera inner leaf extract). In some embodiments, the composition comprises about 0.26 mg/mL nutrient source (e.g., Aloe vera inner leaf extract). In some embodiments, the composition comprises about 0.27 mg/mL nutrient source (e.g., Aloe vera inner leaf extract). In some embodiments, the composition comprises about 0.28 mg/mL nutrient source (e.g., Aloe vera inner leaf extract). In some embodiments, the composition comprises about 0.29 mg/mL nutrient source (e.g., Aloe vera inner leaf extract). In some embodiments, the composition comprises about 0.31 mg/mL nutrient source (e.g., Aloe vera inner leaf extract). In some embodiments, the composition comprises about 0.32 mg/mL nutrient source (e.g., Aloe vera inner leaf extract). In some embodiments, the composition comprises about 0.33 mg/mL nutrient source (e.g., Aloe vera inner leaf extract). In some embodiments, the composition comprises about 0.34 mg/mL nutrient source (e.g., Aloe vera inner leaf extract). In some embodiments, the composition comprises about 0.35 mg/mL nutrient source (e.g., Aloe vera inner leaf extract).

In some embodiments, the composition comprises between about 0.01 w/w % and about 0.05 w/w % nutrient source (e.g., Aloe vera inner leaf extract). In some embodiments, the composition comprises between about 0.02 w/w % and about 0.04 w/w % nutrient source (e.g., Aloe vera inner leaf extract).

In some embodiments, the composition comprises about 0.02 w/w % nutrient source (e.g., Aloe vera inner leaf extract). In some embodiments, the composition comprises about 0.03 w/w % nutrient source (e.g., Aloe vera inner leaf extract). In some embodiments, the composition comprises about 0.04 w/w % nutrient source (e.g., Aloe vera inner leaf extract).

In some embodiments, the composition comprises between about 0.10 mg/mL and about 50 mg/mL magnesium fortification (e.g., MgCl). In some embodiments, the composition comprises between about 0.15 mg/mL and about 40 mg/mL magnesium fortification (e.g., MgCl). In some embodiments, the composition comprises between about 0.20 mg/mL and about 30 mg/mL magnesium fortification (e.g., MgCl). In some embodiments, the composition comprises about 0.25 mg/mL magnesium fortification (e.g., MgCl).

In some embodiments, the composition comprises about 0.20 mg/mL magnesium fortification (e.g., MgCl). In some embodiments, the composition comprises about 0.21 mg/mL magnesium fortification (e.g., MgCl). In some embodiments, the composition comprises about 0.22 mg/mL magnesium fortification (e.g., MgCl). In some embodiments, the composition comprises about 0.23 mg/mL magnesium fortification (e.g., MgCl). In some embodiments, the composition comprises about 0.24 mg/mL magnesium fortification (e.g., MgCl). In some embodiments, the composition comprises about 0.25 mg/mL magnesium fortification (e.g., MgCl). In some embodiments, the composition comprises about 0.26 mg/mL magnesium fortification (e.g., MgCl). In some embodiments, the composition comprises about 0.27 mg/mL magnesium fortification (e.g., MgCl). In some embodiments, the composition comprises about 0.28 mg/mL magnesium fortification (e.g., MgCl). In some embodiments, the composition comprises about 0.29 mg/mL magnesium fortification (e.g., MgCl). In some embodiments, the composition comprises about 0.30 mg/mL magnesium fortification (e.g., MgCl).

In some embodiments, the composition comprises between about 0.01 w/w % and about 0.05 w/w % magnesium fortification (e.g., MgCl). In some embodiments, the composition comprises between about 0.02 w/w % and about 0.04 w/w % magnesium fortification (e.g., MgCl)

In some embodiments, the composition comprises about 0.02 w/w % magnesium fortification (e.g., MgCl). In some embodiments, the composition comprises about 0.03 w/w % magnesium fortification (e.g., MgCl). In some embodiments, the composition comprises about 0.04 w/w % magnesium fortification (e.g., MgCl).

In some embodiments, the composition comprises between about 0.3 mg/mL and about 1.20 mg/mL antioxidant (e.g., ascorbic acid). In some embodiments, the composition comprises between about 0.4 mg/mL and about 1.0 mg/mL antioxidant (e.g., ascorbic acid). In some embodiments, the composition comprises between about 0.5 mg/mL and about 0.80 mg/mL antioxidant (e.g., ascorbic acid). In some embodiments, the composition comprises between about 0.6 and about 0.7 mg/mL antioxidant (e.g., ascorbic acid). In some embodiments, the composition comprises between about 0.6 and about 0.65 mg/mL antioxidant (e.g., ascorbic acid). In some embodiments, the composition comprises about 0.61 mg/mL antioxidant (e.g., ascorbic acid).

In some embodiments, the composition comprises about 0.55 mg/mL antioxidant (e.g., ascorbic acid). In some embodiments, the composition comprises about 0.56 mg/mL antioxidant (e.g., ascorbic acid). In some embodiments, the composition comprises about 0.57 mg/mL antioxidant (e.g., ascorbic acid). In some embodiments, the composition comprises about 0.58 mg/mL antioxidant (e.g., ascorbic acid). In some embodiments, the composition comprises about 0.59 mg/mL antioxidant (e.g., ascorbic acid). In some embodiments, the composition comprises about 0.60 mg/mL antioxidant (e.g., ascorbic acid). In some embodiments, the composition comprises about 0.61 mg/mL antioxidant (e.g., ascorbic acid). In some embodiments, the composition comprises about 0.62 mg/mL antioxidant (e.g., ascorbic acid). In some embodiments, the composition comprises about 0.63 mg/mL antioxidant (e.g., ascorbic acid). In some embodiments, the composition comprises about 0.64 mg/mL antioxidant (e.g., ascorbic acid). In some embodiments, the composition comprises about 0.65 mg/mL antioxidant (e.g., ascorbic acid).

In some embodiments, the composition comprises between about 0.03 w/w % and about 0.12 w/w % antioxidant (e.g., ascorbic acid). In some embodiments, the composition comprises between about 0.03 w/w % and about 0.12 w/w % antioxidant (e.g., ascorbic acid). In some embodiments, the composition comprises between about 0.04 w/w % and about 0.10 w/w % antioxidant (e.g., ascorbic acid). In some embodiments, the composition comprises between about 0.05 w/w % and about 0.08 w/w % antioxidant (e.g., ascorbic acid).

In some embodiments, the composition comprises about 0.05 w/w % antioxidant (e.g., ascorbic acid). In some embodiments, the composition comprises about 0.06 w/w % antioxidant (e.g., ascorbic acid). In some embodiments, the composition comprises about 0.07 w/w % antioxidant (e.g., ascorbic acid). In some embodiments, the composition comprises about 0.08 w/w % antioxidant (e.g., ascorbic acid).

In some embodiments, the composition comprises between about 1.5 mg/mL and about 6.0 mg/mL flavoring agent. In some embodiments, the composition comprises between about 2.0 mg/mL and about 5.0 mg/mL flavoring agent. In some embodiments, the composition comprises between about 2.4 mg/mL and about 3.2 mg/mL flavoring agent.

In some embodiments, the composition comprises about 2.8 mg/mL flavoring agent. In some embodiments, the composition comprises about 2.4 mg/mL flavoring agent. In some embodiments, the composition comprises about 2.5 mg/mL flavoring agent. In some embodiments, the composition comprises about 2.6 mg/mL flavoring agent. In some embodiments, the composition comprises about 2.7 mg/mL flavoring agent. In some embodiments, the composition comprises about 2.8 mg/mL flavoring agent. In some embodiments, the composition comprises about 2.9 mg/mL flavoring agent. In some embodiments, the composition comprises about 3.0 mg/mL flavoring agent. In some embodiments, the composition comprises about 3.1 mg/mL flavoring agent. In some embodiments, the composition comprises about 3.2 mg/mL flavoring agent.

In some embodiments, the composition comprises between about 0.1 w/w % and about 0.6 w/w % flavoring agent. In some embodiments, the composition comprises between about 0.2 w/w % and about 0.4 w/w % flavoring agent.

In some embodiments, the composition comprises about 0.2 w/w % flavoring agent. In some embodiments, the composition comprises about 0.3 w/w % flavoring agent. In some embodiments, the composition comprises about 0.4 w/w % flavoring agent.

Solid Forms

In some embodiments, the composition is a solid (e.g. tablet or powder) which, when mixed together in appropriate amounts with water, achieves a final concentration of all ingredients within consumable and palatable levels.

In some embodiments, the composition comprises, on a weight percent basis, about 92 w/w % to about 96 w/w % water, about 0.1 w/w % to about 0.5 w/w % alkaline buffering agent (e.g. dipotassium phosphate), about 0.01 w/w % to about 0.5 w/w % magnesium fortification (e.g. magnesium chloride), about 0.01 w/w % to about 0.05 w/w % nutrient source (e.g. Aloe vera inner leaf extract), about 4 w/w % to about 8 w/w % carbohydrate source (e.g. agave nectar), about 0.02 w/w % to about 0.1 w/w % antioxidant (e.g. ascorbic acid), and about 0.1 w/w % to about 0.5 w/w % flavoring agent. In some embodiments, the composition comprises, on a weight percent basis, about 93 w/w % water, about 0.3 w/w % alkaline buffering agent (e.g. dipotassium phosphate), about 0.025 w/w % magnesium fortification (e.g. magnesium chloride), about 0.03 w/w % nutrient source (e.g. Aloe vera inner leaf extract), about 6 w/w % carbohydrate source (e.g. agave nectar), about 0.06 w/w % antioxidant (e.g. ascorbic acid), and about 0.3 w/w % flavoring agent.

In some embodiments, the composition comprises, on a mass basis, about 240 parts carbohydrate source (e.g. agave nectar), about 12 parts alkaline buffering agent (e.g. dipotassium phosphate), about 11 parts natural flavor, about 2.4 parts antioxidant (e.g. ascorbic acid), about 1.3 parts nutrient source (e.g. Aloe vera inner leaf extract), and about 1 part magnesium fortification (e.g. magnesium chloride). In some embodiments, the composition comprises, on a mass basis, between about 150 parts to about 400 parts carbohydrate source (e.g. agave nectar), between about 5 parts to about 20 parts alkaline buffering agent (e.g. dipotassium phosphate), between about 5 parts to about 20 parts natural flavor, between about 1 part to about 5 parts antioxidant (e.g. ascorbic acid), between about 0.5 parts to about 2 parts nutrient source (e.g. Aloe vera inner leaf extract), and between about 0.5 parts to about 2 parts magnesium fortification (e.g. magnesium chloride).

In some embodiments, the composition comprises between about 2.5 w/w % and 9.0 w/w % alkaline buffering agent (e.g., dipotassium phosphate). In some embodiments, the composition comprises between about 3.0 w/w % and about 7.0 w/w % alkaline buffering agent (e.g., dipotassium phosphate). In some embodiments, the composition comprises between about 3.5 w/w % and about 6.0 w/w % alkaline buffering agent (e.g., dipotassium phosphate). In some embodiments, the composition comprises between about 4.0 w/w % and about 5.0 w/w % alkaline buffering agent (e.g., dipotassium phosphate). In some embodiments, the composition comprises between about 4.2 w/w % and about 4.8 w/w % alkaline buffering agent (e.g., dipotassium phosphate). In some embodiments, the composition comprises between about 4.3 w/w % and about 4.7 w/w % alkaline buffering agent (e.g., dipotassium phosphate). In some embodiments, the composition comprises between about 4.4 w/w % and about 4.6 w/w % alkaline buffering agent (e.g., dipotassium phosphate). In some embodiments, the composition comprises about 4.5 w/w % alkaline buffering agent (e.g., dipotassium phosphate).

In some embodiments, the composition comprises about 4.0 w/w % alkaline buffering agent (e.g., dipotassium phosphate). In some embodiments, the composition comprises about 4.1 w/w % alkaline buffering agent (e.g., dipotassium phosphate). In some embodiments, the composition comprises about 4.2 w/w % alkaline buffering agent (e.g., dipotassium phosphate). In some embodiments, the composition comprises about 4.3 w/w % alkaline buffering agent (e.g., dipotassium phosphate). In some embodiments, the composition comprises about 4.4 w/w % alkaline buffering agent (e.g., dipotassium phosphate). In some embodiments, the composition comprises about 4.5 w/w % alkaline buffering agent (e.g., dipotassium phosphate). In some embodiments, the composition comprises about 4.6 w/w % alkaline buffering agent (e.g., dipotassium phosphate). In some embodiments, the composition comprises about 4.7 w/w % alkaline buffering agent (e.g., dipotassium phosphate). In some embodiments, the composition comprises about 4.8 w/w % alkaline buffering agent (e.g., dipotassium phosphate). In some embodiments, the composition comprises about 4.9 w/w % alkaline buffering agent (e.g., dipotassium phosphate). In some embodiments, the composition comprises about 5.0 w/w % alkaline buffering agent (e.g., dipotassium phosphate).

In some embodiments, the composition comprises between about 50 w/w % carbohydrate source (e.g., agave nectar) and about 95 w/w % carbohydrate source (e.g., agave nectar). In some embodiments, the composition comprises between about 60 w/w % carbohydrate source (e.g., agave nectar) and about 95 w/w % carbohydrate source (e.g., agave nectar). In some embodiments, the composition comprises between about 70 w/w % carbohydrate source (e.g., agave nectar) and about 95 w/w % carbohydrate source (e.g., agave nectar). In some embodiments, the composition comprises between about 75 w/w % carbohydrate source (e.g., agave nectar) and about 95 w/w % carbohydrate source (e.g., agave nectar). In some embodiments, the composition comprises between about 80 w/w % carbohydrate source (e.g., agave nectar) and about 95 w/w % carbohydrate source (e.g., agave nectar). In some embodiments, the composition comprises between about 85 w/w % carbohydrate source (e.g., agave nectar) and about 95 w/w % carbohydrate source (e.g., agave nectar). In some embodiments, the composition comprises about 90 w/w % carbohydrate source (e.g., agave nectar).

In some embodiments, the composition comprises about 85 w/w % carbohydrate source (e.g., agave nectar). In some embodiments, the composition comprises about 86 w/w % carbohydrate source (e.g., agave nectar). In some embodiments, the composition comprises about 87 w/w % carbohydrate source (e.g., agave nectar). In some embodiments, the composition comprises about 88 w/w % carbohydrate source (e.g., agave nectar). In some embodiments, the composition comprises about 89 w/w % carbohydrate source (e.g., agave nectar). In some embodiments, the composition comprises about 90 w/w % carbohydrate source (e.g., agave nectar). In some embodiments, the composition comprises about 91 w/w % carbohydrate source (e.g., agave nectar). In some embodiments, the composition comprises about 92 w/w % carbohydrate source (e.g., agave nectar). In some embodiments, the composition comprises about 93 w/w % carbohydrate source (e.g., agave nectar). In some embodiments, the composition comprises about 94 w/w % carbohydrate source (e.g., agave nectar). In some embodiments, the composition comprises about 95 w/w % carbohydrate source (e.g., agave nectar).

In some embodiments, the composition comprises between about 0.5 w/w % and about 2.0 w/w % acid source (e.g., ascorbic acid). In some embodiments, the composition comprises between about 0.6 w/w % and about 1.7 w/w % acid source (e.g., ascorbic acid). In some embodiments, the composition comprises between about 0.7 w/w % and about 1.4 w/w % acid source (e.g., ascorbic acid). In some embodiments, the composition comprises between about 0.8 w/w % and about 1.1 w/w % acid source (e.g., ascorbic acid). In some embodiments, the composition comprises about 0.9 w/w % acid source (e.g., ascorbic acid).

In some embodiments, the composition comprises about 0.6 w/w % acid source (e.g., ascorbic acid). In some embodiments, the composition comprises about 0.7 w/w % acid source (e.g., ascorbic acid). In some embodiments, the composition comprises about 0.8 w/w % acid source (e.g., ascorbic acid). In some embodiments, the composition comprises about 0.9 w/w % acid source (e.g., ascorbic acid). In some embodiments, the composition comprises about 1.0 w/w % acid source (e.g., ascorbic acid). In some embodiments, the composition comprises about 1.1 w/w % acid source (e.g., ascorbic acid). In some embodiments, the composition comprises about 1.2 w/w % acid source (e.g., ascorbic acid). In some embodiments, the composition comprises about 1.3 w/w % acid source (e.g., ascorbic acid). In some embodiments, the composition comprises about 1.4 w/w % acid source (e.g., ascorbic acid).

In some embodiments, the composition comprises between about 0.25 w/w % and about 1.0 w/w % nutrient source (e.g., Aloe vera inner leaf extract). In some embodiments, the composition comprises between about 0.30 w/w % and about 0.90 w/w % nutrient source (e.g., Aloe vera inner leaf extract). In some embodiments, the composition comprises between about 0.35 w/w % and about 0.80 w/w % nutrient source (e.g., Aloe vera inner leaf extract). In some embodiments, the composition comprises between about 0.40 w/w % and about 0.70 w/w % nutrient source (e.g., Aloe vera inner leaf extract). In some embodiments, the composition comprises between about 0.40 w/w % and about 0.60 w/w % nutrient source (e.g., Aloe vera inner leaf extract). In some embodiments, the composition comprises about 0.5 w/w % nutrient source (e.g., Aloe vera inner leaf extract).

In some embodiments, the composition comprises about 0.40 w/w % nutrient source (e.g., Aloe vera inner leaf extract). In some embodiments, the composition comprises about 0.42 w/w % nutrient source (e.g., Aloe vera inner leaf extract). In some embodiments, the composition comprises about 0.44 w/w % nutrient source (e.g., Aloe vera inner leaf extract). In some embodiments, the composition comprises about 0.46 w/w % nutrient source (e.g., Aloe vera inner leaf extract). In some embodiments, the composition comprises about 0.48 w/w % nutrient source (e.g., Aloe vera inner leaf extract). In some embodiments, the composition comprises about 0.52 w/w % nutrient source (e.g., Aloe vera inner leaf extract). In some embodiments, the composition comprises about 0.54 w/w % nutrient source (e.g., Aloe vera inner leaf extract). In some embodiments, the composition comprises about 0.56 w/w % nutrient source (e.g., Aloe vera inner leaf extract). In some embodiments, the composition comprises about 0.58 w/w % nutrient source (e.g., Aloe vera inner leaf extract). In some embodiments, the composition comprises about 0.60 w/w % nutrient source (e.g., Aloe vera inner leaf extract).

In some embodiments, the composition comprises between about 0.20 w/w % and about 0.60 w/w % magnesium fortification (e.g., MgCl). In some embodiments, the composition comprises between about 0.25 w/w % and about 0.55 w/w % magnesium fortification (e.g., MgCl). In some embodiments, the composition comprises between about 0.30 w/w % and about 0.50 w/w % magnesium fortification (e.g., MgCl). In some embodiments, the composition comprises between about 0.32 w/w % and about 0.42 w/w % magnesium fortification (e.g., MgCl). In some embodiments, the composition comprises between about 0.34 w/w % and about 0.40 w/w % magnesium fortification (e.g., MgCl). In some embodiments, the composition comprises between about 0.36 w/w % and about 0.38 w/w % magnesium fortification (e.g., MgCl). In some embodiments, the composition comprises about 0.37 w/w % magnesium fortification (e.g., MgCl).

In some embodiments, the composition comprises about 0.32 w/w % magnesium fortification (e.g., MgCl). In some embodiments, the composition comprises about 0.33 w/w % magnesium fortification (e.g., MgCl). In some embodiments, the composition comprises about 0.34 w/w % magnesium fortification (e.g., MgCl). In some embodiments, the composition comprises about 0.35 w/w % magnesium fortification (e.g., MgCl). In some embodiments, the composition comprises about 0.36 w/w % magnesium fortification (e.g., MgCl). In some embodiments, the composition comprises about 0.36 w/w % magnesium fortification (e.g., MgCl). In some embodiments, the composition comprises about 0.38 w/w % magnesium fortification (e.g., MgCl). In some embodiments, the composition comprises about 0.39 w/w % magnesium fortification (e.g., MgCl). In some embodiments, the composition comprises about 0.40 w/w % magnesium fortification (e.g., MgCl). In some embodiments, the composition comprises about 0.41 w/w % magnesium fortification (e.g., MgCl). In some embodiments, the composition comprises about 0.42 w/w % magnesium fortification (e.g., MgCl).

In some embodiments, the composition comprises between about 0.5 w/w % and about 2.0 w/w % antioxidant (e.g., ascorbic acid). In some embodiments, the composition comprises between about 0.6 w/w % and about 1.7 w/w % antioxidant (e.g., ascorbic acid). In some embodiments, the composition comprises between about 0.7 w/w % and about 1.4 w/w % antioxidant (e.g., ascorbic acid). In some embodiments, the composition comprises between about 0.8 w/w % and about 1.1 w/w % antioxidant (e.g., ascorbic acid). In some embodiments, the composition comprises about 0.9 w/w % antioxidant (e.g., ascorbic acid).

In some embodiments, the composition comprises about 0.6 w/w % antioxidant (e.g., ascorbic acid). In some embodiments, the composition comprises about 0.7 w/w % antioxidant (e.g., ascorbic acid). In some embodiments, the composition comprises about 0.8 w/w % antioxidant (e.g., ascorbic acid). In some embodiments, the composition comprises about 0.9 w/w % antioxidant (e.g., ascorbic acid). In some embodiments, the composition comprises about 1.0 w/w % antioxidant (e.g., ascorbic acid). In some embodiments, the composition comprises about 1.1 w/w % antioxidant (e.g., ascorbic acid). In some embodiments, the composition comprises about 1.2 w/w % antioxidant (e.g., ascorbic acid). In some embodiments, the composition comprises about 1.3 w/w % antioxidant (e.g., ascorbic acid). In some embodiments, the composition comprises about 1.4 w/w % antioxidant (e.g., ascorbic acid).

In some embodiments, the composition comprises between about 2 w/w % and about 8 w/w % flavoring agent. In some embodiments, the composition comprises between about 2.5 w/w % and about 7 w/w % flavoring agent. In some embodiments, the composition comprises between about 3 w/w % and about 6 w/w % flavoring agent. In some embodiments, the composition comprises between about 3.5 w/w % and about 5.5 w/w % flavoring agent. In some embodiments, the composition comprises between about 3.5 w/w % and about 5.0 w/w % flavoring agent. In some embodiments, the composition comprises between about 3.5 w/w % and about 4.5 w/w % flavoring agent. In some embodiments, the composition comprises about 4 w/w % flavoring agent.

In some embodiments, the composition comprises about 3.5 w/w % flavoring agent. In some embodiments, the composition comprises about 3.7 w/w % flavoring agent. In some embodiments, the composition comprises about 3.9 w/w % flavoring agent. In some embodiments, the composition comprises about 4.0 w/w % flavoring agent. In some embodiments, the composition comprises about 4.1 w/w % flavoring agent. In some embodiments, the composition comprises about 4.3 w/w % flavoring agent. In some embodiments, the composition comprises about 4.5 w/w % flavoring agent. In some embodiments, the composition comprises about 4.7 w/w % flavoring agent. In some embodiments, the composition comprises about 4.9 w/w % flavoring agent. In some embodiments, the composition comprises about 5.1 w/w % flavoring agent. In some embodiments, the composition comprises about 5.3 w/w % flavoring agent. In some embodiments, the composition comprises about 5.5 w/w % flavoring agent.

### Manufacturing

In some embodiments, the composition is made by mixing dry components with purified water in sterile environment to yield an initial composition, then processing the initial composition through sterilization unit to yield a sterile composition, and packaging the sterile composition in an aseptic facility.

In some embodiments, the composition is packaged by aseptic processing. Aseptic processing is a process by which a sterile (aseptic) product is packaged in a sterile container in a way that maintains sterility. In aseptic processing, products are flash-heated to between 195° F. and 295° F. (i.e. between 91° C. and 146° C.). Aseptic packages typically are, but are not limited to, a material made of paper, polyethylene, and aluminum, with a tight polyethylene layer inside. Aseptic processing can help achieve long shelf lives for food, pharmaceutical, and other products.

In some embodiments, the packaging materials are opaque. In some embodiments, the packaging materials are transparent. In some embodiments, the packaging materials are translucent. In some embodiments, the packaging materials are semi-translucent. In some embodiments, the packaging materials are semi-transparent.

### Shelf-Life

In some embodiments, the composition has a shelf life of greater than 300 days. In some embodiments, the composition has a shelf life of greater than 350 days. In some embodiments, the composition has a shelf life of greater than 400 days. In some embodiments, the composition has a shelf life of greater than 450 days.

### Administration

In some embodiments, the composition is mixed with a suitable amount of a suitable liquid (e.g., water) and administered to a subject in need of rehydration. In some embodiments, the solid dosage form is mixed with a suitable amount of water and administered to a subject in need of rehydration. In some embodiments, the solid dosage form is mixed with 250 mL of water and administered to a subject in need of rehydration. In some embodiments, the solid dosage form is mixed with 500 mL of water and administered to a subject in need of rehydration. In some embodiments, the solid dosage form is mixed with 1 L of water and administered to a subject in need of rehydration. In some embodiments, the solid dosage form is mixed with 2 Ls of water and administered to a subject in need of rehydration. In some embodiments, the solid dosage form is mixed with 3 Ls of water and administered to a subject in need of rehydration.

### Methods of Treatment

Disclosed herein, in some embodiments, are methods of treating or preventing dehydration and/or acidosis comprising administering to an individual in need thereof a composition disclosed herein.

### Dehydration

Disclosed herein, in certain embodiments, are methods of treating or preventing dehydration in a subject in need thereof, comprising administering to the subject an alkaline drink composition disclosed herein. Dehydration occurs when more fluid is lost than is taken in and the body does not have enough water and other fluids to carry out its normal functions. If fluids are not replaced, a person may become dehydrated. Dehydration can commonly be caused by diarrhea, vomiting, fever, sweating, insufficient consumption of liquids especially during hot weather or exercise, prolonged exposure to dry air (e.g. an airplane cabin), prolonged exposure to high altitudes, increased urination (e.g. due to undiagnosed or untreated diabetes mellitus or diabetes insipidus), certain medications (e.g. diuretics, stimulants, antihistamines, blood pressure medications, or some psychiatric medications), and alcohol.

In some embodiments, the subject in need thereof is an infant. In some embodiments, the subject in need thereof is a child. In some embodiments, the subject in need thereof is an adult. In some embodiments, the subject in need thereof is an older adult. In some embodiments, the subject in need thereof is a person with an acute illness. In some embodiments, the subject in need thereof is a person with a cold or a sore throat. In some embodiments, the subject in need thereof is a person with diarrhea. In some embodiments, the subject in need thereof is a person with a chronic illness. In some embodiments, the subject in need thereof is a person with diabetes, kidney disease, alcoholism, or adrenal gland disorders. In some embodiments, the subject in need thereof is an athlete. In some embodiments, the subject in need thereof is an endurance athlete (e.g. distance runner, distance cycler, triathlete, hiker, backpacker, or mountain climber). In some embodiments, the subject in need thereof is a person living, working, or exercising at altitudes above 8,000 feet. In some embodiments, the subject in need thereof is a person working or exercising outside in hot and/or humid weather.

In some embodiments, dehydration results from prolonged physical activity, prolonged exposure to dry air, blood loss or hypotension (e.g., due to physical trauma), diarrhea, hyperthermia, shock, vomiting, burns, lacrimation, use of drugs (e.g., methamphetamines), excessive consumption of alcoholic beverages, infectious diseases (e.g, cholera, gastroenteritis, shigellosis, and yellow fever), malnutrition, electrolyte disturbance (hypernatremia, hyponatremia), fasting, hyperglycemia, glycosuria, uremia, and diabetes insipidus. In some embodiments, dehydration results from prolonged physical activity (e.g., endurance sports, cycling, running, hiking, etc.). In some embodiments, dehydration results from diarrhea.

In some embodiments, the dehydration is associated with sustained physical activity. In some embodiments, the composition is administered on a regular basis with increased consumption in days leading up to physical activity. Ideal dosage is 1 to 2 bottles per day regularly, with up to 4 bottles per day within 5 days of a sustained physical activity. Effects can be seen however by replacing any beverage with the compositions disclosed herein. In this case, customer should drink desired amount (serving size 8 oz) before and during exercise, or at any time for general hydration.

In some embodiments, the dehydration is classified as mild dehydration (e.g., 3%-5%). In some embodiments, the dehydration is classified as moderate dehydration (e.g., 6%-9%). In some embodiments, the dehydration is classified as severe dehydration (e.g., greater than 10%).

In some embodiments, administering a composition disclosed herein alleviates one or more symptoms associated with dehydration, for example headache, hypotension, dizziness, dry or sticky mouth, sleepiness or tiredness, thirst, decreased sweating, decreased urination, constipation dry skin, rapid heartbeat, rapid breathing, fever, or delirium.

### Acidic Diet

Disclosed herein, in certain embodiments, are methods of treating or preventing a disease, disorder, or condition that is induced by an acidic diet in a subject in need thereof, comprising administering to the subject an alkaline drink composition disclosed herein. In some embodiments, the disease, disorder, or condition induced by an acidic diet is the formation dental caries and tooth decay. In some embodiments, the disease, disorder, or condition induced by an acidic diet is kidney stones. In some embodiments, the disease, disorder, or condition induced by an acidic diet is weakening of bones or osteoporosis. In some embodiments, the disease, disorder, or condition induced by an acidic diet is weakening of joints or arthritis. In some embodiments, the disease, disorder, or condition induced by an acidic diet is weakening of muscles. In some embodiments, the disease, disorder, or condition induced by an acidic diet is heart disease. In some embodiments, the disease, disorder, or condition induced by an acidic diet is kidney disease. In some embodiments, the disease, disorder, or condition induced by an acidic diet is high blood pressure. In some embodiments, the disease, disorder, or condition induced by an acidic diet is back pain.

### EXAMPLES

### Example 1

### Alkaline Drink Preparation

An alkaline drink is prepared as follows: at least one alkaline buffering agent and at least one magnesium fortification are separately dissolved in water and then blended. Next, at least one nutrient source is added and dissolved completely. Next, at least one carbohydrate source is added and dissolved completely. Next, at least one antioxidant is added and dissolved completely. Next, at least one flavoring agent is added and dissolved completely. The composition has a buffered pH of at least 7.1. The drink is packaged and distributed.

### Example 2

### Alkaline Drink Preparation

An alkaline drink is prepared as follows: dipotassium phosphate and magnesium chloride are separately dissolved in water and then blended, in amounts to result in final concentrations of about 3.1 mg/mL and about 0.25 mg/mL, respectively. Next, Aloe vera inner leaf extract powder is added in an amount to result in a final concentration of about 0.33 mg/mL and dissolved completely. Next, agave nectar is added in an amount to result in a final concentration of about 61 mg/mL and dissolved completely. Next, ascorbic acid is added in an amount to result in a final concentration of about 0.61 mg/mL and dissolved completely. Next, grape and strawberry flavoring agents are added in an amount to result in a final concentration of about 2.8 mg/mL and dissolved completely. The composition has a buffered pH of about 7.4. The drink is packaged and distributed.

### Example 3

### Concentrated Alkaline Drink Preparation

An alkaline drink is prepared in concentrated form. Without water, ingredients are combined in the following concentrations: 0.37 w/w % magnesium chloride, 4.5 w/w % dipotassium phosphate, 0.48 w/w % Aloe vera inner leaf extract powder, 90 w/w % agave nectar, 0.90 w/w % ascorbic acid, and 4.1 w/w % flavoring. The combined ingredients are packaged and distributed. A consumer mixes a serving of the concentrated form with water and drinks it

### Example 4

### Unsuccessful Alkaline Drink Preparation

An alkaline drink was prepared as follows: sodium bicarbonate (as an alkaline buffering agent) and at least one magnesium fortification were separately dissolved in water and then blended. Next, at least one nutrient source was added and dissolved completely. Next, at least one carbohydrate source was added and dissolved completely. Next, at least one antioxidant was added and dissolved completely. Next, at least one flavoring agent was added and dissolved completely. The drink was packaged, but as the product sat on the shelf the pH increased beyond the desired range.

### Example 5

### Treatment of Dehydration Due to Gastroenteritis

A person suffers gastroenteritis and experiences symptoms including nausea, vomiting, and diarrhea, and fever. After a period of time, they begin experiencing symptoms of dehydration. The person consumes the drink slowly until their urine becomes clear in color. They recover from their dehydration.

### Example 6

### Treatment of Dehydration Due to Uncontrolled Diabetes Mellitus

A person has undiagnosed or uncontrolled diabetes mellitus. Due to increased thirst and increased urination as symptoms of the disease, the person begins experiencing symptoms of dehydration. The person consumes about 2 liters of the drink per day. Their dehydration improves.

### Example 7

### Prevention of Dehydration Due to Exercise

A runner competes in a marathon on a hot and humid day. Prior, during, and after the race, the runner consumes the drink. The runner suffers no effects from dehydration.

### Example 8

### Prevention of Symptoms of Altitude Sickness

A traveler goes to Colorado and spends several days at altitudes above 8,000 feet. They experience symptoms including nausea, fatigue, and headache. The traveler begins consuming up to 4 bottles per day of the alkaline drink. Their symptoms improve.

### Example 9

### Prevention of Tooth Decay

An athlete regularly consumes 2 bottles per day of a conventional acidic sports drink. They are advised by their dentist of acid damage to their tooth enamel and formation of dental caries. They stop consuming their conventional sports drink and begin consuming 2 bottles per day of the alkaline drink. Further tooth decay is prevented.

### Example 10

### Athletic Performance Enhancement

An athlete is preparing for a competition. The athlete drinks one beverage per day for 30 days leading up to, or 4 beverages per day for 5 days leading up to their competition. The athlete experiences more efficient oxygen delivery to their muscles and neutralization of acidic metabolic waste. The athlete outperforms their previous personal physical limitations.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition, comprising:
(a) at least one alkaline buffering agent;
(b) at least one acid source;
(c) at least one carbohydrate source;
(d) at least one nutrient source; and
(e) at least one magnesium fortification; and
wherein the composition has a pH between about 7.1 to about 7.7.

2. The composition of claim 1, wherein the at least one alkaline buffering agent comprises monopotassium phosphate, dipotassium phosphate, tripotassium phosphate, monosodium phosphate, disodium phosphate, trisodium phosphate, potassium bicarbonate, sodium bicarbonate, or any combinations thereof.

3. The composition of claim 1, wherein the at least one acid source comprises ascorbic acid, citric acid, acetic acid, carbonic acid, or any combination thereof.

4. The composition of claim 1, wherein the at least one carbohydrate source comprises at least one low glycemic index carbohydrate source selected from the group consisting of: agave nectar, coconut nectar, coconut palm sugar, lucuma powder, honey, maple syrup, yacon syrup, date sugar, brown rice syrup, barley malt syrup, blackstrap molasses, cane juice, sorghum syrup, lactose, galactose, fructose, mogrosides (a.k.a. luo han guo or Siraitia grosvenorii extract), sugar alcohol, erythritol, mannitol, isomalt, lactitol, sorbitol, glycerol, xylitol, maltitol, HSH, arabitol, sweet dietary fiber, inulin, oligofructose, xylooligosaccharide, or any combinations thereof.

5. The composition of claim 1, wherein the at least one nutrient source comprises Aloe vera inner leaf extract, coconut water, green tea, black tea, white tea, tea, fruit juice, vegetable juice, or any combination thereof.

6. The composition of claim 1, wherein the at least one magnesium fortification comprises magnesium chloride, magnesium citrate, magnesium aspartate, magnesium lactate, magnesium glycinate, magnesium sulfate, magnesium oxide, magnesium hydroxide, or any combination thereof.

7. The composition of claim 1, further comprising at least one antioxidant selected from the group consisting of: ascorbic acid (Vitamin C), vitamin A, vitamin E, beta-carotene, other carotenoids, flavonoids, phenolic acids, curcumin, flavonoligans, xanthones, eugenol, capsaicin, bilirubin, citric acid, oxalic acid, phytic acid, uric acid, R-alpha-lipoic acid, N-Acetylcysteine, or any combination thereof.

8. The composition of claim 1, wherein the composition is a beverage for human consumption.

9. The composition of claim 1, wherein the composition is a liquid.

10. The composition of claim 9, comprising between about 2.5 mg/mL and about 3.5 mg/mL alkaline buffering agent.

11. The composition of claim 9, comprising between about 0.2 w/w % and about 0.4 w/w % alkaline buffering agent.

12. The composition of claim 9, comprising between about 0.4 mg/mL and about 1.0 mg/mL acid source.

13. The composition of claim 9, comprising between about 0.04 w/w % and about 0.10 w/w % acid source.

14. The composition of claim 9, comprising between about 50 mg/mL and about 70 mg/mL carbohydrate source.

15. The composition of claim 9, comprising between about 4 w/w % and about 10 w/w % carbohydrate source.

16. The composition of claim 9, comprising between about 0.25 mg/mL and about 0.40 mg/mL nutrient source.

17. The composition of claim 9, comprising between about 0.01 w/w % and about 0.05 w/w % nutrient source.

18. The composition of claim 9, comprising between about 0.1 mg/mL and about 0.5 mg/mL magnesium fortification.

19. The composition of claim 9, comprising between about 0.01 w/w % and about 0.05 w/w % magnesium fortification.

20. The composition of claim 1, wherein the composition is a solid.

21. The composition of claim 20, comprising between about 4.0 w/w % and about 5.0 w/w % alkaline buffering agent.

22. The composition of claim 20, comprising between about 75 w/w % and about 95 w/w % carbohydrate source.

23. The composition of claim 20, comprising between about 0.3 w/w % and about 0.7 w/w % nutrient source.

24. The composition of claim 20, comprising between about 0.2 w/w % and about 0.6 w/w % magnesium fortification.

25. The composition of claim 20, comprising between about 0.5 w/w % and about 2.0 w/w % antioxidant.

26. The composition of claim 1, wherein the composition has a shelf life of greater than 350 days.

27. The composition of claim 1, wherein the composition has a shelf life of greater than 450 days.

28. A composition, comprising:
(a) at least one alkaline buffering agent;
(b) at least one acid source; and
(c) at least one carbohydrate source;
wherein the composition has a pH between about 7.1 to about 7.7.

29. The composition of claim 28, wherein the at least one alkaline buffering agent comprises monopotassium phosphate, dipotassium phosphate, tripotassium phosphate, monosodium phosphate, disodium phosphate, trisodium phosphate, potassium bicarbonate, sodium bicarbonate, or any combinations thereof.

30. The composition of claim 28, wherein the at least one acid source comprises ascorbic acid, citric acid, acetic acid, carbonic acid, or any combination thereof.

31. The composition of claim 28, wherein the at least one carbohydrate source comprises at least one low glycemic index carbohydrate source selected from the group consisting of: agave nectar, coconut nectar, coconut palm sugar, lucuma powder, honey, maple syrup, yacon syrup, date sugar, brown rice syrup, barley malt syrup, blackstrap molasses, cane juice, sorghum syrup, lactose, galactose, fructose, mogrosides (a.k.a. luo han guo or Siraitia grosvenorii extract), sugar alcohol, erythritol, mannitol, isomalt, lactitol, sorbitol, glycerol, xylitol, maltitol, HSH, arabitol, sweet dietary fiber, inulin, oligofructose, xylooligosaccharide, or any combinations thereof.

32. The composition of claim 28, wherein the composition is a beverage for human consumption.

33. The composition of claim 28, wherein the composition is a liquid.

34. The composition of claim 33, comprising between about 2.5 mg/mL and about 3.5 mg/mL alkaline buffering agent.

35. The composition of claim 33, comprising between about 0.2 w/w % and about 0.4 w/w % alkaline buffering agent.

36. The composition of claim 33, comprising between about 50 mg/mL and about 70 mg/mL carbohydrate source.

37. The composition of claim 33, comprising between about 4 w/w % and about 10 w/w % carbohydrate source.

38. The composition of claim 28, wherein the composition is a solid.

39. The composition of claim 38, comprising between about 4.0 w/w % and about 5.0 w/w % alkaline buffering agent.

40. The composition of claim 38, comprising between about 0.5 w/w % and about 2.0 w/w % acid source.

41. The composition of claim 38, comprising between about 75 w/w % and about 95 w/w % carbohydrate source.

42. The composition of claim 28, further comprising at least one of: a nutrient source, antioxidant, magnesium fortification, and a flavoring agent.

43. The composition of claim 28, wherein the composition has a shelf life of greater than 350 days.

44. The composition of claim 28, wherein the composition has a shelf life of greater than 450 days.

45. A method of treating or preventing dehydration in an individual in need thereof, comprising administering to the individual a composition of any of claims 1-44.

46. The method of claim 45, wherein the cause of dehydration is prolonged physical activity, prolonged exposure to dry air, blood loss or hypotension (e.g., due to physical trauma), diarrhea, hyperthermia, shock, vomiting, burns, lacrimation, use of drugs (e.g., methamphetamines), excessive consumption of alcoholic beverages, infectious diseases (e.g, cholera, gastroenteritis, shigellosis, and yellow fever), malnutrition, electrolyte disturbance (hypernatremia, hyponatremia), fasting, hyperglycemia, glycosuria, uremia, diabetes insipidus, or any combination thereof.

47. A method of alleviating symptom(s) of dehydration in an individual in need thereof, comprising administering to the individual a composition of any of claims 1-44.

48. The method of claim 47, wherein the symptom comprises headache, hypotension, dizziness, dry or sticky mouth, sleepiness or tiredness, thirst, decreased sweating, decreased urination, constipation dry skin, rapid heartbeat, rapid breathing, fever, delirium, or any combination thereof.
